(19) United States
(12) Patent Application Publication
(10) Pub. No.: US 2026/0275308 A1
(43) Pub. Date: Sep. 17, 2026

(54) ALKALINE PHOSPHATASE FOR USE IN TREATING ACUTE-ON-CHRONIC KIDNEY INJURY

(71) Applicant: AM-PHARMA B.V., Utrecht (NL)

(72) Inventors: Matthias Johannes WINKEL, Utrecht (NL); Erik Jan VAN DEN BERG, Utrecht (NL); Maarten Cornelis KRAAN, Utrecht (NL); Sharon Eva RICHARDS, Utrecht (NL)

(21) Appl. No.: 19/167,192

(22) PCT Filed: Mar. 21, 2024
(86) PCT No.: PCT/NL2024/050139

§ 371 Date: Sep. 19, 2025

(30) Foreign Application Priority Data
Mar. 21, 2023 (EP) 23163281.1

(51) Int. Cl. C12N 9/16; A61K 38/00; A61P 13/12

(52) U.S. Cl. CPC C12N 9/16; A61P 13/12; A61K 38/00; C12Y 301/03001

(57) ABSTRACT

The present disclosure relates to the use of alkaline phosphatases, and in particular improved alkaline phosphatases such as RecAP, for the prevention, treatment, cure, or amelioration of the symptoms of acute kidney injury on top of chronic kidney injury. The application relates to alkaline phosphatase for use in methods of preserving renal function, shortening the duration of renal replacement therapy, preserving the creatinine clearance, decreasing the risk of death in subjects with acute kidney injury on top of chronic kidney injury.

### BACKGROUND

The kidneys perform several functions in an animal body, such as excretion of waste, acid-base homeostasis, osmolality regulation, blood pressure regulation and hormone secretion. To enable the kidneys to perform these tasks, the kidneys receive, despite their relatively small size, approximately 20% of the cardiac output. Consequently, a disruption of blood flow to the kidneys (renal blood flow, RBF) has a direct impact on many of the functions of the kidney. For instance, reduction of excretion of nitrogenous waste and disturbances of fluid and electrolyte balances could then occur. On the long term, reduced RBF, but also other toxic events, such as ischemia (reperfusion injury), use of contrast media or (other) nephrotoxic drugs, e.g. antibiotics, can be so stressful to the kidneys that it they results in acute kidney injury (AKI).

Acute Kidney Injury (AKI) is observed in up to 60% of patients in intensive care units (ICUs), and its incidence is increasing (Hoste et al. Intensive Care Med 2015; 41:1411-23; Hoste et al. Critical Care 2006; 10:R73; Nisula et al. Intensive Care Med 2013; 39:420-8; Vincent et al. Critical Care Medicine 2006; 34:344-53). Development of AKI in sepsis patients is associated with an increased mortality (Kellum et al. Critical Care Medicine 2016; 193:281-7), while survivors are at risk of developing chronic kidney injury (CKI, also termed Chronic Kidney Disease (CKD)) (Chawla et al. Kidney Int 2011; 79:1361-9; Oppert et al. Nephrol Dial Transplant 2008; 23:904-9; Vaara et al. Crit Care 2012; 16: R197) resulting in a tremendous burden for both patient and society.

AKI itself is a common disorder, with a population incidence of about 2,000 per million population (pmp). Patients with CKI, as evidenced by a low estimated glomerular filtration rate (eGFR) or by the presence of proteinuria for more than 3 months, are at higher risk for developing AKI. CKI is a strong risk factor for cardiovascular events, and patients with CKI are at particular mortality risk if they develop AKI on top of CKI, called acute-on-chronic kidney injury (AoCKI).

It is very important to prevent and/or adequately treat AoCKI, because first, AKI is accompanied with high costs, morbidity and mortality in CKI patients, and second, AoCKI may lead to a further decrease in renal function in these already impaired patients, possibly leading to irreversible loss of renal function below a critical threshold.

### SUMMARY OF THE INVENTION

The present invention provides an alkaline phosphatase (AP) for use in a method to treat acute kidney injury (AKI) in a subject in need thereof, comprising administering an effective amount of AP to said subject, wherein the subject has acute-on-chronic kidney injury (AoCKI), and wherein said chronic kidney injury (CKI) is mild, moderate or severe, preferably moderate to severe. Preferably, the pre-AKI estimated glomerular filtration rate (eGFR) is ≤75 ml/min/1.73 m², such as ≤60 ml/min/1.73 m², more preferably ≤45 ml/min/1.73 m². The pre-AKI eGFR preferably is ≥15 ml/min/1.73 m², such as ≥25 ml/min/1.73 m².

In a preferred embodiment, AP for use according to the invention is provided, wherein the administration of AP results in a decreased risk of developing major adverse kidney events by day 90 (MAKE90). Preferably the decrease in risk of MAKE90 comprises a decrease in risk of MAKE90 with respect to the risk of MAKE90 in the absence of treatment. Preferably, MAKE90 comprises one or more of the following events: (i) death before or on day 90, (ii) renal replacement therapy before or at day 90, (iii) ≥25% drop in eGFR at day 90, relative to pre-AKI eGFR, and (iv) rehospitalization before or at day 90.

In a preferred embodiment, the invention provides an AP for use according to the invention, wherein the AP is administered in at least one 500 U/kg to 2,000 U/kg dose.

In a preferred embodiment, the invention provides an AP for use according to the invention, wherein the AP is a human AP.

In a preferred embodiment, the invention provides an AP for use according to the invention, wherein the AP is a recombinant AP, preferably wherein the recombinant AP is chimeric, more preferably wherein the chimeric AP has at least 80%, at least 85%, at least 90%, at least 95%, at least 96%, at least 97%, at least 98%, at least 99%, or 100% sequence identity to the amino acid sequence of RecAP (SEQ ID NO: 1).

In a preferred embodiment, the invention provides an AP for use according to the invention, wherein AP is administered once daily or in three daily doses.

In a preferred embodiment, the invention provides an AP for use according to the invention, wherein AP is administered intravenously.

In a preferred embodiment, the invention provides an AP for use according to the invention, wherein the AP dose is about 1.6 mg/kg of RecAP and/or about 1000 U/kg of RecAP.

In a preferred embodiment, the invention provides an AP for use according to the invention, wherein the administration of at least one dose of AP results in the preservation at day 90 of glomerular filtration rate (GFR) or estimated GFR (eGFR) in the subject relative to the pre-AKI eGFR.

In a preferred embodiment, the invention provides an AP for use according to the invention, wherein the AKI is due to or accompanied by sepsis. This is called sepsis-associated AKI (SA-AKI).

In a preferred embodiment, the invention provides an AP for use according to the invention, wherein treatment is initiated within 24 hours after sepsis is detected and/or within 48 hours after acute kidney injury (AKI) is detected in said subject.

In a preferred embodiment, the invention provides an AP for use according to the invention, wherein a decreased eGFR due to chronic kidney injury has been determined at least 15 days, preferably at least 30 days, more preferably at least 60 days, most preferably at least 90 days prior to the administration of AP.

### BRIEF DESCRIPTION OF THE DRAWINGS

FIG. 1 provides an oversight on the individual trial visit flow of the subjects, including the screening and Baseline period (prior day 1), the treatment period (day 1 to day 3)) and the Follow-up period (day 4 to day 180).

FIG. 2 shows a RecAP amino acid sequence (SEQ ID NO: 1).

FIG. 3 is a flowchart, showing the enrolment, randomization, non-treated patients, the efficacy analysis as per protocol, the safety analysis population, as well as the combined population. Abbreviations: ICF (Informed Consent Form), ITT (Intent To Treat), mCKD (“moderate to severe” Chronic Kidney Disease).

FIG. 4 is a Kaplan Meier Plot showing the survival rates for subject treated with ilofotase alfa versus patients treated with placebo. The survival rates at day 90 are compared with the Wald test for differences in proportions and a respective p-value is given. The mortality rate can be calculated 1-survival rate. Proportions of patients alive at day 90: Ilofotase alpha: 0.6554. Placebo: 0.6397. P-value based in Wald test for difference in proportions of patients alive at day 90 (Ilofotase alpha—Placebo: 0.3404. Patients that died after day 90 were censored at day 90.

FIG. 5 is a Kaplan Meier Plot showing the proportion of subjects without an MAKE90 event for subject treated with ilofotase alfa versus patients treated with placebo. The rates of subjects without a MAKE90 event at day 90 are compared with the Wald test for differences in proportions and a respective p-value is given. The rate of subjects with an MAKE90 event until day 90 can be calculated as 1-proportion of subjects without an MAKE90 event. Proportion of patients without MAKE90 at day 90: Ilofotase alpha: 0.4277. Placebo: 0.3533. P-value for difference (Ilofotase alpha-Placebo: 0.030705. Patients that withdrew prior to day 90 without a MAKE90 event were censored at the day of withdrawal. Patients that withdrew, completed the trial, died, or were hospitalized after day 90 were censored at day 90. MAKE90 events (on RRT at day 90/<25% drop of in eGFR) were recorded on visits up to 12 days prior or after day 90. Abbreviations: MAKE90 (Major Adverse kidney events until day 90); RRT (Renal Replacement Therapy).

FIG. 6 is a plot, showing the predicted probability of experiencing a Make90 event including the 95% confidence interval. The predictions have been calculated based on the parameter estimates of the logistic regression model described in Table 7. Predicted possibilities are taken from the results of the logistic regression model which included MAKE90 as the outcome of interest, and treatment, pre-AKI reference eGFR, and pre-AKI reference eGFR by treatment interaction (p=0.0235). Patients who did not meet the criteria for having a confirmed MAKE90 event are assumed not to have a MAKE90 event in the logistic regression model.

FIG. 7 is a Kaplan Meier Plot showing the proportion of subjects with pre-AKI reference eGFR <60 without an MAKE90 event for subject treated with ilofotase alfa versus patients treated with placebo. The rates of subjects without a MAKE90 event at day 90 are compared with the Wald test for differences in proportions and a respective p-value is given. The rate of subject s with an MAKE90 event until day 90 can be calculated as 1-proportion of subjects without an MAKE90 event. Proportion of patients without MAKE90 at day 100: Ilofotase alpha: 0.4032. Placebo: 0.2092. P-value for difference (Ilofotase alpha-Placebo: 0.004357. Abbreviations: MAKE90 (Major Adverse kidney events until day 90).

FIG. 8 is a Kaplan Meier Plot showing the proportion of subjects with pre-AKI reference eGFR <75 without an MAKE90 event for subject treated with ilofotase alfa versus patients treated with placebo. The rates of subjects without a MAKE90 event at day 90 are compared with the Wald test for differences in proportions and a respective p-value is given. The rate of subject s with an MAKE90 event until day 90 can be calculated as 1-proportion of subjects without an MAKE90 event. Proportion of patients without MAKE90 at day 90: Ilofotase alpha: 0.4361. Placebo: 0.2971. P-value for difference (Ilofotase alpha-Placebo: 0.0047. Patients that withdrew prior to day 90 without a MAKE90 event were censored at the day of withdrawal. Patients that withdrew, completed the trial, died, or were hospitalized after day 90 were censored at day 90. MAKE90 events (on RRT at day 90/<25% drop of in eGFR) were recorded on visits up to 12 days prior or after day 90. Abbreviations: MAKE90 (Major Adverse kidney events until day 90); RRT (Renal Replacement Therapy).

### DETAILED DESCRIPTION OF THE INVENTION

### I General

Sepsis is the leading cause of acute kidney injury (AKI) and a major cause of death. Patients with sepsis-associated AKI (SA-AKI) have a high mortality and morbidity and are at risk of developing or worsening existing chronic kidney injury (CKI), also termed chronic kidney disease (CKD). Alkaline phosphatase (AP) is a homodimeric endogenous enzyme present in many cells and organs, e.g., intestines, placenta, liver, bone, kidney, and granulocytes. It exerts detoxifying effects through dephosphorylation of endotoxins; pathogen associated molecular pattern molecules (PAMPS e.g., lipopolysaccharide [LPS]) and damage-associated molecular pattern molecules (DAMPS e.g., adenosine tri- and di-phosphate [ATP and ADP]). In animal models of sepsis and AKI, administration of AP attenuates the inflammatory response, improves renal function, and/or reduces mortality.

Sepsis-associated AKI (SA-AKI) is a multifactorial syndrome with inflammatory, nephrotoxic, and ischemic insults occurring simultaneously with other pathophysiological responses rapidly leading to renal impairment (Bonventre et al. J Clin Invest 2011; 121:4210-21; Gomez et al. Shock 2014; 41:3-11). Currently, no approved pharmacologic interventions are available to prevent or treat AKI (Joannidis et al. Intensive Care Medicine 2017; 43:730-49).

Alkaline phosphatase (AP) was originally considered as a novel treatment for sepsis in general (Poelstra et al. American Journal of Pathology 1997; 151:1163-69; Verweij et al. Shock 2004; 22:174-9). In two small clinical trials bovine AP administration improved renal function in patients with sepsis (Heemskerk et al. Crit Care Med 2009; 37:417-23, e1; Pickkers et al. Crit Care 2012; 16: R14).

A novel, recombinant chimeric human AP medicinal product, called recAP was used as an intravenous (IV) infusion for the treatment of SA-AKI in a Phase 2 trial STOP-AKI. Herein, a survival benefit was observed in the two highest dose groups, 0.8 mg/kg and 1.6 mg/kg groups, compared to the placebo group. No safety or tolerability concerns were observed for any of the doses tested (0.4, 0.8 and 1.6 mg/kg). The 1.6 mg/kg recAP dose was selected for a REVIVAL Phase 3 trial based on the significant survival benefit observed.

In the STOP-AKI trial, which is the subject of patent publication WO2019172766, a history of CKI was one of the exclusion criteria. In the REVIVAL trial, however, subjects having mild to moderate CKI, prior to developing AKI were not excluded. The REVIVAL trial thus not only treated subjects having normal or mildly impaired kidney function (i.e., eGFR >60 ml/min/1.73 m² prior to being hospitalized) as well as subjects already having a reduced renal function (i.e., eGFR≤60 ml/min/1.73 m²) due to moderate to severe chronic kidney disease prior to being hospitalized. The latter are even more susceptible to further acute renal decline, called acute-on-chronic kidney injury (AoCKI), leaving the subject with an even lower rest kidney function after the acute part of the kidney injury has been treated. The present invention surprisingly shows that the treatment of this selected patient population, that was not included in the STOP-AKI trial, benefited more from treatment with AP than those patients that did not suffer from CKI, prior to acquiring AKI.

Acute kidney injury (AKI) itself is a common disorder, with a population incidence of about 2,000 per million population (pmp). Patients with CKI, as evidenced by a low eGFR or presence of proteinuria for more than 3 months, are at higher risk for developing AKI. CKI is a strong risk factor for cardiovascular events, and patients with CKI are at particular mortality risk if they develop AoCKI.

RecAP is a chimeric AP that combines the properties of two human isoenzymes, intestinal and placental AP (Kiffer-Moreira et al. PLoS One 2014; 9: e89374). Replacing the crown domain of intestinal AP (the most biologically active isoenzyme) with the crown domain of placental AP (which has the longest half-life) creates a highly stable, biologically active enzyme (Kiffer-Moreira et al. PLoS One 2014; 9: e89374). See, e.g., U.S. Pat. Nos. 8,586,032 and 8,557,545, and U.S. Patent Appl. Publ. Nos. US20170009216 and US20160250299.

Accordingly, the present invention relates to an AP for use in a method to treat acute kidney injury (AKI) in a subject in need thereof, comprising administering an effective amount of AP to said subject, wherein the subject has acute-on-chronic kidney injury (AoCKI), and wherein the chronic kidney injury (CKI) is mild, moderate, or severe, preferably moderate to severe.

### Definitions

In this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Wherever aspects are described herein with the language “comprising,” otherwise analogous aspects described in terms of “consisting of” and/or “consisting essentially of” are also provided.

The term “about” as used in connection with a numerical value throughout the specification and the claims denotes an interval of accuracy, familiar and acceptable to a person skilled in the art. In general, such interval of accuracy is +15%.

Unless defined otherwise, all technical and scientific terms used herein have the same meaning as commonly understood by one of ordinary skill in the art to which this disclosure is related.

Units, prefixes, and symbols are denoted in their Système International de Unites (SI) accepted form.

Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value is explicitly recited, it is to be understood that values which are about the same quantity or amount as the recited value are also within the scope of the invention. Where a combination is disclosed, each sub-combination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

As used herein the terms “treat,” “treatment,” or “treatment of” refers to (i) reducing a potential or risk for a disease or disorder, e.g., AKI (e.g., SA-AKI and/or AoCKI), (ii) reducing the occurrence of a disease or disorder, e.g., AKI (e.g., SA-AKI and/or AoCKI), (iii) reducing the severity (e.g., ameliorating the symptoms) of a disease or disorder, e.g., AKI (e.g., SA-AKI and/or AoCKI), (iv) reducing the risk of a major adverse kidney event by day 90 (MAKE90), or (v) a combination thereof. For example, treating can refer to the ability of a therapy when administered to a subject, to prevent or reduce the risk of kidney injury, e.g., SA-AKI and/or AoCKI, from occurring (for example, in subjects diagnosed with sepsis or at a risk for sepsis) and/or to cure or to alleviate symptoms, signs, or causes of kidney injury, e.g., SA-AKI and/or AoCKI. The term treating also refers to mitigating or decreasing at least one clinical symptom and/or inhibition or delay in the progression of the condition and/or prevention or delay of the onset of a disease or illness when compared with a non-treated (or placebo treated) group. Thus, the terms “treat,” “treating” or “treatment of” (or grammatically equivalent terms) refer to both prophylactic and therapeutic treatment regimes. In case of renal disease, it is preferred that subjects, after being treated with an AP with a method as disclosed herein improve in renal function or show less decline in renal function, in particular at day 90 after the start of the treatment, relative to subjects that have not been treated with said AP.

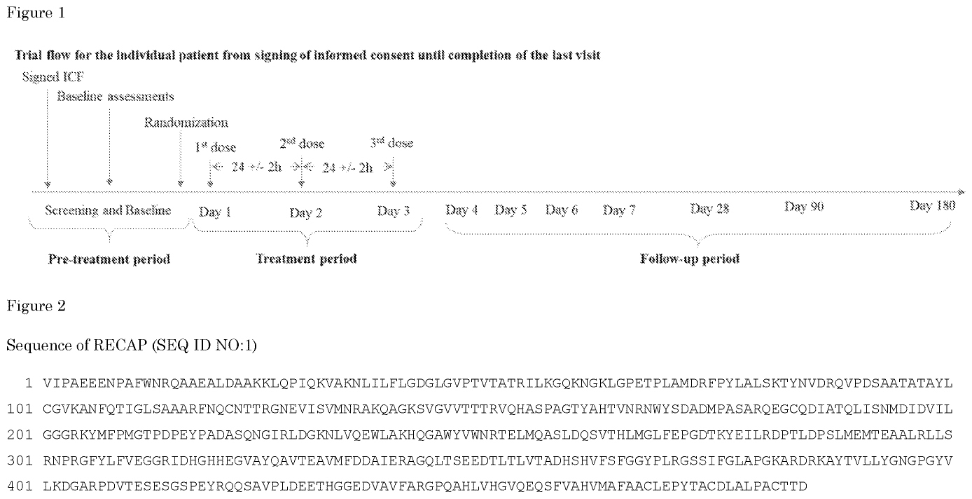

As used herein, the term “day” in the context of a numeric value and in relation to the treatment, such as “day 90” or “day 28”, the day count relative to the day of administration of the first dose of either AP or placebo (being day 1) is meant (see also FIG. 1).

As used herein, the term “preserving” includes preventing a reduction, slowing down a reduction, stopping a reduction and/or at least partly reversing a reduction of a renal function.

The term “increasing” is not necessarily limited to increasing renal function to a value equal to or higher than that before said treatment occurred. It includes partly restoring renal function.

The term “treatment with a risk of decreasing renal function” is typically used to refer to a treatment which bears the risk that renal function is reduced by said treatment when comparing the value of at least one renal-related parameter to a recognized or average (laboratory) value of said parameter, or by comparing said parameter to the value before said treatment is performed. If, for example, the amount of protein in the urine of a subject, preferably a human being, is significantly above a recognized or average (laboratory) value, said renal function is said to be “decreased.” The corresponding analysis can be performed in a laboratory but also in a home setting. For example, since September 2006 the Dutch “Nierstichting” has introduced a simple test (named Kidney check (“Niercheck”)) which can be performed at home to test whether the kidneys function properly. This test is for instance directed to the amount of protein in the urine.

The terms “subject” or “patient” as used herein refer to any subject, particularly a mammalian subject, for whom therapy or prognosis of kidney injury, e.g., SA-AKI and/or AoCKI is desired. As used herein, the terms “subject” or “patient” include any human or nonhuman animal. As used herein, phrases such as “a patient having AKI, preferably SA-AKI, and/or AoCKI” or a “patient having sepsis” includes subjects, such as mammalian subjects, that would benefit from the administration of a therapy with AP, as disclosed herein.

In some aspects of the present disclosure, a subject is a naïve subject. A naïve subject is a subject that has not been administered a therapy, for example a therapeutic agent related to kidney functioning. In some aspects, a naïve subject has not been treated with a therapeutic agent prior to being diagnosed with kidney injury, e.g., SA-AKI and/or AoCKI, or a disease or condition (e.g., sepsis) that can lead to kidney injury.

In another aspect, a subject has received therapy and/or one or more doses of a therapeutic agent prior to being diagnosed as having kidney injury, e.g., SA-AKI and/or AoCKI, or a disease or condition (e.g., sepsis) that can lead to kidney injury.

In some aspects, a subject can be administered at least one therapeutically effective dose of an AP, e.g., RecAP, if the subject's pre-AKI eGFR is below a predetermined threshold level, or if the pre-AKI eGFR is within a predetermined range.

The terms “therapeutic agent” and “drug” as used herein also refer to any therapeutically active substance that is administered to a subject having a disease or disorder, e.g., kidney injury, e.g., SA-AKI and/or AoCKI, or a disease or condition (e.g., sepsis) that can lead to kidney injury to produce a desired, usually beneficial, effect. A therapeutic agent can also be a pro-drug, which metabolizes into the desired therapeutically active substance when administered to a subject. In some aspects, the therapeutic agent is a prophylactic agent. In addition, a therapeutic agent can be pharmaceutically formulated. A therapeutic agent can also be or comprise a radioactive isotope or agent activated by some other form of energy such as light or ultrasonic energy, or by other circulating molecules that can be systemically administered.

In some aspects of the present disclosure, a therapeutic agent for use in a method of treatment, prevention, or amelioration of the symptoms or long-term adverse effects, such as MAKE90, of kidney injury, e.g., SA-AKI and/or AoCKI, or a disease or condition (e.g., sepsis) that can lead to kidney injury, can comprise an AP, e.g., RecAP; alone or in combination with one or more standard therapeutic agents generally used for the treatment of kidney injury, e.g., SA-AKI and/or AoCKI, or a disease or condition (e.g., sepsis) that can lead to kidney injury.

A “therapeutically effective” amount as used herein is an amount of therapeutic agent that provides some improvement or benefit to a subject having a disease or disorder, e.g., kidney injury, e.g., SA-AKI and/or AoCKI, or a disease or condition (e.g., sepsis) that can lead to kidney injury. Thus, a “therapeutically effective” amount is an amount that provides some alleviation, mitigation, and/or decrease in at least one clinical symptom or adverse event, such as MAKE90, of a disease or disorder, e.g., kidney injury, e.g., SA-AKI and/or AoCKI, or a disease or condition (e.g., sepsis) that can lead to kidney injury.

Clinical symptoms associated with kidney injury, e.g., SA-AKI and/or AoCKI, or a disease or condition (e.g., sepsis) that can lead to kidney injury that can be treated by the compositions, methods, as specific dosage regimens of the disclosure are well known to those skilled in the art. Further, those skilled in the art will appreciate that the therapeutic effects need not be complete or curative, as long as some benefit is provided to the subject. In some aspects, the term “therapeutically effective” refers to an amount of a therapeutic agent therapeutic agent that is capable of altering biomarker levels, e.g., endogenous creatinine clearance (ECC) or eGFR in a patient in need thereof.

As used herein, a “sufficient amount” or “an amount sufficient to achieve a particular result” in a patient having a disease or disorder, e.g., kidney injury, e.g., SA-AKI and/or AoCKI, or a disease or condition (e.g., sepsis) that can lead to kidney injury refers to an amount of a therapeutic agent (e.g., an AP such as RecAP) that is effective to produce a desired effect, which is optionally a therapeutic effect (i.e., by administration of a therapeutically effective amount). In some aspects, such particular result is an improvement in kidney function, an increase in survival, and/or a reduction in risk of experiencing an adverse effect, such as MAKE90.

As used herein, the term “healthcare provider” refers to individuals or institutions that directly interact and administer to a living subject, e.g., a human patient. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for examination or processing of materials derived from a living subject, e.g., a human being. Non-limiting examples of processing include biological, biochemical, serological, chemical, immuno-hematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

### II. Treatment of Acute Kidney Injury with AP

In certain aspects, the present disclosure is related to alkaline phosphatase (AP) for use in methods for preserving or improving kidney function in populations of subjects, in particular, patients suffering from CKI, that have been determined to respond particularly well to treatment with AP.

The REVIVAL study has identified statistically significant correlations between thresholds corresponding to degrees of severity of CKI and clinical outcome of AP treatment. As discussed above, stratifying patients in the REVIVAL clinical trial according to pre-AKI eGFR has identified particular effects of AP administration on specific subgroups. The parameter or parameters defining each of these subgroups can be used, e.g., to personalize AP therapy to specific subgroups, to select patients for treatment, to make decisions related to the AP treatment (e.g., modify dosing or dosage schedule), and/or to evaluate the likelihood of a positive outcome.

Accordingly, in some aspects, the AP for use in methods disclosed herein relates to the administration of AP for treating AKI in a subject determined to already have mild, moderate or severe CKI, comprising administering an AP such as RecAP to the subject.

As used herein, the term “moderate chronic kidney injury”, refers to chronic kidney damage or impairment of kidney function resulting in a creatinine clearance rate between 45-60 ml/min/1.73 m².

As used herein, the term “severe chronic kidney injury” refers to chronic kidney damage or impairment of kidney function resulting in a creatinine clearance rate lower than 45 ml/min/1.73 m².

As used herein, the term “moderate to severe chronic kidney injury” refers to chronic kidney damage or impairment of kidney function resulting in an eGFR of from 15 ml/min/1.73 m² to 60 ml/min/1.73 m², preferably an eGFR from 25 ml/min/1.73 m² to 60 ml/min/1.73 m².

As used herein, the term “mild kidney injury” refers to kidney damage or impairment of kidney function resulting in a creatinine clearance rate of higher than 60 ml/min/1.73 m², preferably between 60 ml/min/1.73 m² and 75 ml/min/1.73 m².

As used herein the term “pre AKI eGFR” refers to the estimated glomerular filtration rate before onset of AKI and which is already decreased due to CKI. Typically, eGFR of CKI patients is known and determined regularly. In some aspects, the decreased eGFR due to chronic kidney injury, also referred to within this disclosure as pre-AKI eGFR, has been determined at least 15 days, preferably at least 30 days, more preferably at least 60 days, most preferably at least 90 days prior to the administration of AP. eGFR is an estimation of the actual glomerular filtration rate and is widely used as measurement of kidney function.

In some aspects, the present disclosure provides alkaline phosphatase (AP) for use in a method to treat acute kidney disease injury in a subject in need thereof, wherein the method comprises administering an effective amount of alkaline phosphatase (AP) to said subject, wherein the pre-AKI eGFR is ≤75 ml/min/1.73 m², preferably ≤60 ml/min/1.73 m². In some aspects, the pre-AKI eGFR is ≤45 ml/min/1.73 m². In some aspects the pre-AKI eGFR is ≥15 ml/min/1.73 m² or ≥25 ml/min/1.73 m². In some aspects, the pre-AKI is between 15-60 ml/min/1.73 m² or between 25-60 ml/min/1.73 m². In some aspects, the pre-AKI is between 15-45 ml/min/1.73 m² or between 25-45 ml/min/1.73 m².

In some aspects, the AP (e.g., RecAP) is administered as doses of at least about 500 U/kg, at least about 600 U/kg, at least about 700 U/kg, at least about 800 U/kg, at least about 900 U/kg, at least about 1000 U/kg, at least about 1100 U/kg, at least about 1200 U/kg, at least about 1300 U/kg, at least about 1400 U/kg, at least about 1500 U/kg, at least about 1600 U/kg, at least about 1700 U/kg, at least about 1800 U/kg, at least about 1900 U/kg, or at least about 2000 U/kg per dose. In some aspects, the AP (e.g. RecAP) is administered at a dose above 2000 U/kg per dose. In some aspects, the AP (e.g., RecAP) is administered at a dose below 500 U/kg per dose.

In some aspects, the AP (e.g., RecAP) is administered at a dose between about 500 U/kg and about 1500 U/kg, between about 600 U/kg and about 1400 U/kg, between about 700 U/kg and about 1300 U/kg, between about 800 U/kg and about 1200 U/kg, or between about 900 U/kg and about 1100 U/kg. In some specific aspects, AP is administered as an about 1000 U/kg dose.

In some aspects, the AP is a human AP. In some aspects, the AP is a recombinant AP. In some aspects, the AP is a chimeric AP. In a particular aspect, the chimeric AP is RecAP (SEQ ID NO: 1). In some aspects, an AP as provided herein has at least about 70%, at least about 75%, at least about 80%, at least about 85%, at least about 90%, at least about 95%, at least about 96%, at least about 97%, at least about 98%, or at least about 99% sequence identity to the full length amino acid sequence of SEQ ID NO:1. In some aspects, the AP is a functional fragment (i.e., a fragment of the AP, e.g., AP conserving at least about 10%, at least about 20%, at least about 30%, at least 40%, at least about 50%, at least about 60%, at least 70%, at least about 80%, or at least about 90% of the AP activity of the corresponding full length AP). In some aspects, the AP is a variant or a derivative of an AP disclosed herein. Other AP that can be used are discussed in detail below.

In some aspects, the AP is RecAP (e.g., the clinical grade RecAP used in the present disclosure), and it is administered at a dose of at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg, at least about 1.1 mg/kg, at least about 1.3 mg/kg, at least about 1.4 mg/kg, at least about 1.5 mg/kg, at least about 1.6 mg/kg, at least about 1.7 mg/kg, at least about 1.8 mg/kg, at least about 1.9 mg/kg, at least about 2 mg/kg, at least about 2.1 mg/kg, at least about 2.2 mg/kg, at least about 2.3 mg/kg, or at least about 2.4/kg per dose. In some aspects, the AP is administered as doses above 2.4 mg/kg per dose.

In some aspects, the AP is RecAP (e.g., the clinical grade RecAP used in the present disclosure), and it is administered at a dose of at least about 100 U/kg, at least about 200 U/kg, at least about 300 U/kg, at least about 400 U/kg, at least about 500 U/kg, at least about 600 U/kg, at least about 700 U/kg, at least about 800 U/kg, at least about 900 U/kg, at least about 1000 U/kg, at least about 1100 U/kg, at least about 1200 U/kg, at least about 1300 U/kg, at least about 1400 U/kg, at least about 1500 U/kg, at least about 1600 U/kg, at least about 1700 U/kg, at least about 1800 U/kg, at least about 1900 U/kg, or at least about 2000 U/kg. In some specific aspects, the AP is RecAP, and it is administered at a dose between 500 U/kg to 2000 U/kg.

In some aspects, the AP is RecAP (e.g., the clinical grade RecAP used in the present disclosure) and it is administered at a dose between about 0.8 mg/kg and about 2.4 mg/kg, between about 0.9 mg/kg and about 2.3 mg/kg, between about 1 mg/kg and about 2.2 mg/kg, between about 1.1 mg/kg and about 2.1 mg/kg, between about 1.2 mg/kg and about 2 mg/kg, between about 1.3 mg/kg and about 1.9 mg/kg, between about 1.4 mg/kg and about 1.8 mg/kg, or between about 1.5 mg/kg and about 1.7 mg/kg. In some specific aspects, AP is administered as about 1.6 mg/kg doses.

In some aspects, the AP is RecAP (e.g., the clinical grade RecAP used in the present disclosure) and has a specific activity of at least about 100 U/mg, at least about 200 U/mg, at least about 300 U/mg, at least about 400 U/mg, at least about 500 U/mg, at least about 600 U/mg, at least about 700 U/mg, at least about 800 U/mg, at least about 900 U/mg, at least about 1000 U/mg, at least about 1100 U/mg, at least about 1200 U/mg, at least about 1300 U/mg, at least about 1400 U/mg, at least about 1500 U/mg, at least about 1600 U/mg, at least about 1700 U/mg, at least about 1800 U/mg, at least about 1900 U/mg, or at least about 2000 U/mg.

In some aspects, the AP is RecAP (e.g., the clinical grade RecAP used in the present disclosure) and has a specific activity of about 1000 U per 1.6 mg.

In some aspects, the AP is RecAP and has a specific activity between about 600 U/mg and about 700 U/mg, or between about 500 U/mg and about 800 U/mg, or between about 400 U/mg and about 900 U/mg, or between about 300 U/mg and about 1000 U/mg, or between about 200 U/mg and about 1100 U/mg, or between 100 U/mg and about 1200 U/mg. In some aspects, the AP is RecAP and has a specific activity below 100 U/mg. In some aspects, the AP is RecAP and has a specific activity above 1200 U/mg.

In some aspects, only one dose of AP (e.g., RecAP) is administered per treatment (e.g., one dose per day for 1-7 days). In other aspects, more than one dose of AP is administered. In some aspects, two, three, four, five, six, seven, eight, nine, 10, 11, 12, 13, 14, 15, 16, 17, 18, 19, 20, or 21 doses of AP are administered (e.g., at least two doses per day for 1-7 days).

In some aspects, the AP doses are administered daily. In other aspects, AP doses are administered every 2, 3, 4, 5, 6 or 7 days.

In some aspects, a single dose is administered every day. In some aspects, 2, 3, or more doses are administered every day.

In some aspects, the treatment with AP is less than about 4 days. In some aspects, the treatment with AP is less than 3 days, such as less than 2 days, or less than 1 day.

In a particular aspect, the AP is administered as a daily dosage of about 1000 U/kg dose administered at 3 consecutive days. In some particular aspects, when the AP is RecAP, the AP is administered as a daily 1.6 mg/kg dose administered for 3 consecutive days.

In some aspects, each AP dose, e.g., RecAP (e.g., the clinical grade RecAP used in the present disclosure) dose, is between about 0.10 mg/kg and about 3 mg/kg, or between about 0.20 mg/kg and about 2.9 mg/kg, or between about 0.3 mg/kg and about 2.8 mg/kg, or between about 0.4 mg/kg and about 2.7 mg/kg, or between about 0.5 mg/kg and about 2.6 mg/kg, or between about 0.6 mg/kg and about 2.5 mg/kg, or between about 0.7 mg/kg and about 2.4 mg/kg, or between about 0.8 mg/kg and about 2.3 mg/kg, or between about 0.9 mg/kg and about 2.2 mg/kg, or between about 1 mg and about 2.1 mg/kg, or between about 1.1 mg/kg and about 2 mg/kg, or between about 1.2 mg/kg and about 1.9 mg/kg, or between about 1.3 mg/kg and about 1.8 mg/kg, or between about 1.4 mg/kg and about 1.7 mg/kg.

In some aspects, each AP dose, e.g., RecAP, dose, comprises at least about 0.1 mg AP/kg, at least about 0.2 mg AP/kg, at least about 0.3 mg AP/kg, at least about 0.4 mg AP/kg, at least about 0.5 mg AP/kg, at least about 0.6 mg AP/kg, at least about 0.7 mg AP/kg, at least about 0.8 mg AP/kg, at least about 0.9 mg AP/kg, at least about 1 mg AP/kg, at least about 1.1 mg AP/kg, at least about 1.2 mg AP/kg, at least about 1.3 mg AP/kg, at least about 1.4 mg AP/kg, at least about 1.5 mg AP/kg, at least about 1.6 mg AP/kg, at least about 1.7 mg AP/kg, at least about 1.8 mg AP/kg, at least about 1.9 mg AP/kg, at least about 2 mg AP/kg, at least about 2.1 mg AP/kg, at least about 2.2 mg AP/kg, at least about 2.3 mg AP/kg, at least about 2.4 mg AP/kg, at least about 2.5 mg AP/kg, at least about 2.6 mg AP/kg, at least about 2.6 mg AP/kg, at least about 2.7 mg AP/kg, at least about 2.8 mg AP/kg, at least about 2.9 mg AP/kg, or at least about 3 mg AP/kg.

The AP may be administered via different routes, for example intravenously, rectally, bronchially or orally. In a preferred embodiment, the AP is administered intravenously, e.g., via intravenous injection or infusion.

Although short term preservation of renal function can have immediate life-saving consequences, it is preferred that the effect of AP on the renal function is long lasting.

eGFR can be determined by methods known in the art. Either the Modification of Diet in Renal Disease (MDRD) Study equation or the Chronic Kidney Disease Epidemiology Collaboration (CKD-EPI) equation can be used, preferably the CKD-EPI equation. A skilled person is aware how to estimate eGFR according to either method, for instance by using a website for calculation of the eGFR as provided, for instance, by the national kidney foundation (kidney.org).

In a preferred aspect, the administration of AP results in a decreased risk of developing major adverse kidney events by day 90 (MAKE90), wherein preferably the decrease in risk of MAKE90 comprises a decrease in risk of MAKE90 with respect to the risk of MAKE90 in the absence of treatment.

MAKE90 is a clinically meaningful endpoint for patients with renal disease as it is predictive for poor long-term outcome for patients, including the development of end-stage renal disease (ESRD). The MAKE90 composite components are death, need for dialysis, substantial kidney function deterioration (≥25% decline in estimated glomerular filtration rate (eGFR)) by day 90, and rehospitalization.

Preferably, the MAKE90 comprise one or more of the following events: (i) death before or on day 90, (ii) renal replacement therapy until and including day 28, or at day 90, (iii) ≥25% drop in eGFR at day 90 after treatment with AP started relative to pre-AKI eGFR, and (iv) rehospitalization before or at day 90.

Said one or more events may include (i) death before or on day 90; (ii) renal replacement therapy before or at day 90; (iii) ≥25% drop in eGFR at day 90 after treatment with AP started relative to pre-AKI eGFR; (iv) rehospitalization before or at day 90; (i) death before or on day 90, and (ii) renal replacement therapy before or at day 90; (i) death before or on day 90, and (iii) ≥25% drop in eGFR at day 90 after treatment with AP started relative to pre-AKI eGFR; (i) death before or on day 90, and (iv) rehospitalization before or at day 90; (ii) renal replacement therapy before or at day 90, and (iii) ≥25% drop in eGFR at day 90 after treatment with AP started relative to pre-AKI eGFR; (ii) renal replacement therapy before or at day 90, and (iv) rehospitalization before or at day 90; (iii) ≥25% drop in eGFR at day 90 after treatment with AP started relative to pre-AKI eGFR, and (iv) rehospitalization before or at day 90; (i) death before or on day 90, (ii) renal replacement therapy before or at day 90, and (iii) ≥25% drop in eGFR at day 90 after treatment with AP started relative to pre-AKI eGFR; (i) death before or on day 90, (ii) renal replacement therapy before or at day 90, and (iv) rehospitalization before or at day 90; or (ii) renal replacement therapy before or at day 90, (iii) ≥25% drop in eGFR at day 90 after treatment with AP started relative to pre-AKI eGFR, and (iv) rehospitalization before or at day 90.

In some embodiments, AP (e.g., RecAP) is administered to the subject only if sepsis has been detected less than 96 hours prior initiating the treatment. In other aspects, AP is administered only if sepsis was detected less than 72 hours prior to AKI detection.

In some aspects of the present disclosure, treatment with AP is initiated within 48 hours or within 24 hours after sepsis is detected. The presence of sepsis can be detected, e.g., as disclosed in the Examples section of this application.

In some aspects, treatment is initiated within 48 hours, preferably within 24 hours after AKI is detected in the subject.

In some aspects, the administration of at least one dose of AP results in a shortening of duration or cessation of renal replacement therapy (RRT) in a subject undergoing RRT.

In some aspects, the administration of at least one dose of AP results in the preservation or increase of glomerular filtration rate (GFR) in the subject. GFR can be assessed in several ways, e.g., by inulin or chromium EDTA clearance, or an approximation of GFR can be made, for instance by calculating endogenous creatinine clearance (ECC). This is calculated from a measured 24 hour urine volume, the urine creatinine level, and the serum creatinine level. The GFR can also be estimated (eGFR), based on serum creatinine.

In some aspects disclosed herein, the administration of AP results in an increase of renal function or prevents the reduction of renal function below a critical threshold which would preclude the administration of a certain treatment, e.g., the administration of an antibiotic to treat sepsis. Accordingly, in some aspects, AP administration is able to prevent reduction of renal function below a critical threshold and thus enables such person to receive the treatment. Thus, in some aspects, an indicator of renal function (e.g., ECC or eGFR) is determined prior to administering AP for preserving renal function in order to determine the risk that the renal function of said person is reduced below a certain threshold level.

In some aspects, the invention comprises detecting changes in markers of kidney function, e.g., ECC, eGFR, or blood urea nitrogen (BUN) clearance, alone or in combination with the detection of changes in the levels of one, two, three, or more biomarkers.

In some aspects, the invention comprises predicting an increased clinical response to therapy with AP, e.g., RecAP, based on detected kidney function parameters (e.g., pre-AKI eGFR). In some aspects, the present disclosure comprises evaluating whether a kidney function parameter (e.g., pre-AKI eGFR) falls within a certain range, or it's above or below a certain threshold (e.g., eGFR threshold for severity of chronic kidney injury). Thus, if, e.g., the kidney function parameter (e.g., pre-AKI eGFR), alone or in combination with other biomarkers, indicates that the patient will benefit from therapy with AP, then therapy could commence, or be maintained, or be modified (e.g., increasing or decreasing dosage, or increasing or decreasing frequency of doses).

Conversely, if, e.g., the kidney function parameter (e.g., pre-AKI eGFR), alone or in combination with other biomarkers, indicates that the patient will not benefit from therapy with AP, then therapy could be discontinued, temporarily suspended, modified (e.g., increasing or decreasing dosage or increasing or decreasing frequency of doses), etc.

In other words, specific levels of a kidney function parameter (e.g., pre-AKI eGFR) alone or in combination with other molecular or clinical biomarkers are correlated with clinical efficacy of AP therapy and useful to predict clinical outcomes in specific populations of patients suffering from sepsis and/or AKI on top of CKI.

The invention thus provides alkaline phosphatase (AP) for use in a method to treat acute kidney injury (AKI) in a subject in need thereof, comprising administering an effective amount of AP to said subject, wherein the subject has acute-on-chronic kidney injury, and wherein said chronic kidney injury (CKI) is moderate to severe. In a preferred embodiment, the subject has pre-AKI eGFR between 15-60 ml/min/1.73 m². In a more preferred embodiment, the subject has pre-AKI eGFR between 25-60 ml/min/1.73 m². With pre-AKI eGFR is meant herewith that the eGFR is decreased due to chronic kidney injury and is preferably determined at least 15 days, preferably at least 30 days, more preferably at least 60 days, most preferably at least 90 days prior to administration of AP.

In a preferred embodiment, an AP for use according to the invention is provided, wherein the AP is a human AP.

In a preferred embodiment, the AP is a recombinant AP, preferably chimeric, more preferably having at least 80%, at least 85%, at least 90%, at least 95%, at least 96%, at least 97%, at least 98%, at least 99%, or at least 100% sequence identity to the amino acid sequence of RecAP (SEQ ID NO: 1). Said sequence identity preferably is determined over the full length protein as depicted in SEQ ID NO:1.

In a preferred embodiment, an AP for use according to the invention is provided, wherein the administration of AP leads to a decreased risk of developing major adverse kidney events by day 90 (MAKE90), wherein preferably the decrease in risk of MAKE90 comprises a decrease in risk of MAKE90 with respect to the risk of MAKE90 in the absence of treatment.

In a preferred embodiment, an AP for use according to the invention is provided, wherein sepsis is detected less than 96 hours prior to AP administration.

In a preferred embodiment, an AP for use according to the invention is provided, wherein the sepsis is detected less than 72 hours prior to AKI detection. Preferably, treatment is initiated within 24 hours after sepsis is detected and/or after AKI is detected. In preferred aspects of the invention, AKI is sepsis-associated AKI (SA-AKI). SA-AKI is used to describe AKI that is due to or accompanied by sepsis.

In a preferred embodiment, AP for use according to the invention is provided, wherein AP is administered once daily. In a preferred embodiment, AP is administered intravenously. In a preferred embodiment, AP is administered in three daily doses.

In a preferred embodiment, AP for use according to the invention is provided, wherein the AP is RecAP and the dose is between 0.06 mg/kg and 3.2 mg/kg, or between 375 U/kg and 2,000 U/kg of RecAP, preferably between 0.08 mg/kg and 3.2 mg/kg or between 500 U/kg and 2,000 U/kg.

In a preferred embodiment, AP for use according to the invention is provided, wherein the administration of at least one dose of AP results in a shortening of duration or cessation of renal replacement therapy (RRT) in a subject undergoing RRT.

In one preferred embodiment, AP for use according to the invention is provided, wherein the administration of at least one dose of AP results in the preservation or increase of glomerular filtration rate (GFR) in the subject.

The term “biomarker” as used herein refers to a factor that is a distinctive indicator of a biological process, biological event, and/or pathologic condition, e.g., a predictor of clinical response to treatment with AP, e.g., RecAP. As used herein, the term biomarker encompasses both clinical markers and molecular biomarkers (biological markers). Thus, in the context of the present disclosure, the term “biomarker” encompasses, e.g., “biological biomarkers” or “molecular biomarkers.” In some aspects, the biological or molecular biomarkers used to evaluate kidney function comprise markers of liver function (e.g., alanine aminotransferase, aspartate aminotransferase, gamma-glutamyl transferase, bilirubin, or LDH), C-reactive protein (CRP), interleukin 6 (IL-6), interleukin 18 (IL-18), lipopolysaccharide binding protein, kidney injury molecule 1 (KIM-1) or combinations thereof.

As disclosed above, the term “biomarker” also encompasses “clinical biomarkers,” also referred to as “clinical status markers,” that can be predictive of response to biological therapies, for example, gender, age, concomitant drugs, smoking status, body mass index (BMI), etc.

As discussed above, eGFR that is decreased due to chronic kidney injury (referred to as pre-AKI eGFR) is used for a cut-off approach. Thus, if a subject's pre-AKI eGFR is below 60 ml/min/1.73 m² that subject would become a candidate for treatment with a certain AP therapy, e.g., therapy with a certain AP regimen, for example comprising one or more doses of RecAP.

The AP for use in methods disclosed herein include prescribing, initiating, and/or altering prophylaxis and/or treatment, e.g., for AKI, based at least in part on a subject's historical eGFR (or another kidney function parameter), alone or in combination with one or more additional biomarkers. With historical is meant that the eGFR has been determined prior to the patient being admitted to the hospital, e.g., at least 15, 30, 60 or 90 day before administration of AP is started.

The present disclosure provides a method of determining whether to treat a patient having AoCKI with a therapeutic regimen comprising the administration of an AP wherein the method comprises: (a) obtaining pre-AKI eGFR values from the patient and (b) treating or instructing a healthcare provider to treat the patient, or suspending the treatment, not initiating the treatment, denying the treatment, or instructing a healthcare provider to suspend, not initiate, or deny the treatment with a therapeutic regimen comprising the administration of an AP, e.g., RecAP, if the patient is determined to have higher or lower pre-AKI eGFR compared to predetermined threshold level or levels. Also provided is a method of determining whether a patient having AKI is likely to respond to a therapeutic regimen comprising the administration of an AP, wherein the method comprises: (a) obtaining pre-AKI eGFR values from the patient and (b) determining that the patient is likely to respond, or is not likely to respond, to the treatment with a therapeutic regimen comprising the administration of an AP, e.g., RecAP, if the patient is determined to have higher or lower pre-AKI eGFR compared to the predetermined threshold level or levels.

In one aspect, the disclosure provides a method of determining whether to treat a patient having AoCKI with a therapeutic regimen comprising the administration of an AP, wherein the method comprises: (a) measuring or instructing a clinical laboratory to measure eGFR and optionally levels of additional biomarkers such as kidney injury molecule 1 (KIM-1) in a sample taken from the patient, and (b) treating or instructing a healthcare provider to treat the patient with a therapeutic regimen comprising the administration of an AP if the patient is determined to have lower or decreased eGFR, and higher or increased levels of at least one optional additional biomarker such as KIM-1 in the sample compared to a predetermined biomarker threshold level or levels, or compared to a biomarker level or levels in one or more controls.

Also provided is a method of determining whether a patient having AKI, preferably sepsis associated AKI (SA-AKI) and/or AoCKI, is likely to respond to a therapeutic regimen comprising the administration of an AP, wherein the method comprises: (a) measuring or instructing a clinical laboratory to measure eGFR and optionally levels of additional biomarkers such as KIM-1 in a sample taken from the patient, and (b) determining that the patient is likely to respond to a therapeutic regimen comprising the administration of an AP if the patient is determined to have lower or decreased eGFR, and higher or increased levels of at least one optional additional biomarker such as KIM-1 in the sample compared to a predetermined biomarker threshold level or levels, or compared to a biomarker level or levels in one or more controls.

In one aspect, the disclosure provides a method of determining whether to treat a patient having AKI, preferably sepsis associated AKI (SA-AKI) and/or AoCKI, with a therapeutic regimen comprising the administration of an AP wherein the method comprises (a) obtaining pre-AKI eGFR values from the patient, and (b) suspending the treatment, not initiating treatment, denying the treatment, or instructing a healthcare provider to suspend, not initiate, or deny the treatment of the patient with a therapeutic regimen comprising the administration of an AP, e.g., RecAP, to the patient if the patient is determined to have higher or increased pre-AKI eGFR compared to a predetermined biomarker threshold level or levels.

Also provided is a method of determining whether to treat a patient having AKI, preferably sepsis associated AKI (SA-AKI) and/or AoCKI, with a therapeutic regimen comprising the administration of an AP wherein the method comprises (a) obtaining pre-AKI eGFR values from the patient, and (b) determining that the patient is unlikely to respond to a therapeutic regimen comprising the administration of an AP, e.g., RecAP, to the patient if the patient is determined to have higher or increased pre-AKI eGFR compared to a predetermined biomarker threshold level or levels.

Also provided is a method of selecting a patient diagnosed with AKI, preferably sepsis associated AKI (SA-AKI) and/or AoCKI, as a candidate for treatment with an AP, comprising (a) obtaining pre-AKI eGFR values from the patient, and (b) treating or instructing a healthcare provider to treat the patient with an AP if the patient is determined to have lower or decreased pre-AKI eGFR compared to a predetermined threshold level or levels.

Also provided is a method of selecting a patient diagnosed with AKI, preferably sepsis associated AKI (SA-AKI) and/or AoCKI, as a candidate for treatment with an AP, comprising (a) obtaining pre-AKI eGFR values from the patient, and (b) determining that the patient is likely to respond to a treatment with AP if the patient is determined to have lower or decreased pre-AKI eGFR compared to a predetermined threshold level or levels.

Also provided is a method of selecting a patient diagnosed with AKI, preferably sepsis associated AKI (SA-AKI) and/or AoCKI, as a candidate for treatment with an AP comprising (a) obtaining pre-AKI eGFR values from the patient, and (b) suspending the treatment, not initiating treatment, denying the treatment, or instructing a healthcare provider to suspend, not initiate, or deny the treatment of the patient with an AP, e.g., RecAP, to the patient if the patient is determined to have higher or increased pre-AKI eGFR compared to a predetermined threshold level or levels.

Also provided is a method of selecting a patient diagnosed with AKI, preferably sepsis associated AKI (SA-AKI) and/or AoCKI, as a candidate for treatment with an AP comprising (a) obtaining pre-AKI eGFR values from the patient, and (b) determining that the patient is unlikely to respond to treatment with an AP, e.g., RecAP, if the patient is determined to have higher or increased pre-AKI eGFR compared to a predetermined threshold level or levels. The predetermined eGFR threshold level is about 60 ml/min/1.73 m².

Pre-AKI eGFR values can be obtained from, e.g., a patient medical record. The term “medical record” or “patient medical record” refers to an account of a patient's examination and/or treatment that typically includes one or more of the following: the patient's medical history and complaints, the physician's physical findings, the results of diagnostic tests and procedures, and patient medications and therapeutic procedures. A medical record is typically made by one or more physicians and/or physicians' assistants and it is a written, transcribed or otherwise recorded record and/or history of various illnesses or injuries requiring medical care, and/or inoculations, and/or allergies, and/or treatments, and/or prognosis, and/or frequently health information about parents, siblings, and/or occupation. The record may be reviewed by a physician in diagnosing the condition.

The medical record can be in paper form and/or can be maintained in a computer-readable medium. The medical record can be maintained by a laboratory, physician's office, a hospital, a healthcare maintenance organization, an insurance company, and/or a personal medical record website.

In some aspects, a diagnosis, based at least in part on the measured eGFR, is recorded on or in a medical alert article such as a card, a worn article, and/or a radiofrequency identification (RFID) tag. As used herein, the term “worn article” refers to any article that can be worn on a subject's body, including, but not limited to, a tag, bracelet, necklace, arm band, or head band.

As used herein, the term “diagnosis” means detecting a disease or determining the stage or degree of a disease. Usually, a diagnosis of a disease is based on the evaluation of one or more factors and/or symptoms that are indicative of the disease. That is, a diagnosis can be made based on the presence, absence or amount of a factor which is indicative of presence or absence of the disease or disorder. Each factor or symptom that is considered to be indicative for the diagnosis of a particular disease does not need be exclusively related to the particular disease, e.g. there may be differential diagnoses that can be inferred from a diagnostic factor or symptom. Likewise, there may be instances where a factor or symptom that is indicative of a particular disease is present in an individual that does not have the particular disease.

The term “diagnosis” also encompasses determining the therapeutic effect of a drug therapy, e.g., AP therapy, or predicting the pattern of response to a drug therapy. The diagnostic methods may be used independently, or in combination with other diagnosing and/or staging methods known in the medical arts for a particular disease.

As used herein, the term “differential diagnosis” refers to the determination of which of two or more diseases with similar symptoms is likely responsible for a subject's symptom(s), based on an analysis of the clinical data. The term is also used to refer to the determination of whether a patient is susceptible to treatment with an AP depending on whether the measured eGFR in a patient sample is above or below a predetermined threshold level, or elevated or decreased relative to the level in one or more controls.

The term “prognosis” as used herein refers to a prediction of the probable course and outcome of a clinical condition or disease, e.g., sepsis or SA-AKI. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “favorable prognosis” and “positive prognosis,” or “unfavorable prognosis” and “negative prognosis” as used herein are relative terms for the prediction of the probable course and/or likely outcome of a condition or a disease, e.g., sepsis or SA-AKI. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include increased kidney function, preservation of kidney function, increase in ECC or eGFR (or another kidney function parameter), a reduced risk on adverse events, such as MAKE90, and the like.

The disclosure provides a method of treating a patient having AoCKI, or AP for use in a method of treating AoCKI in a subject, wherein the method comprises: administering an AP to the patient if the patient is determined to have a lower or decreased pre-AKI eGFR compared to predetermined eGFR threshold level.

The disclosure also provides AP for use in a method of treating a patient having AoCKI, wherein the method comprises: (a) obtaining pre-AKI eGFR values from the patient, and (b) administering an AP to the patient if the patient has a lower or decreased pre-AKI eGFR compared to a predetermined eGFR threshold level.

Also provided is AP for use in a method of treating a patient having AoCKI, wherein the method comprises: (a) obtaining pre-AKI eGFR values from the patient, and (b) suspending or not initiating the administration of AP, e.g., RecAP, to the patient if the patients has a higher or increased pre-AKI eGFR compared to a predetermined eGFR threshold level.

The disclosure also provides AP for use in a method of treating a patient having AKI, preferably sepsis associated AKI (SA-AKI) and/or AoCKI, wherein the method comprises: (a) obtaining pre-AKI eGFR values from the patient, and (b) determining whether pre-AKI eGFR values are higher or increased, or lower or decreased compared to a predetermined eGFR threshold level.

In some aspects the method further comprises administering or advising a healthcare provider to administer an AP, e.g., RecAP, to the patient if the patient is determined to have a lower or decreased pre-AKI eGFR compared to a predetermined eGFR threshold level; or to suspend or deny the administration of an AP if the patient is determined to have a higher or increased pre-AKI eGFR level compared to a predetermined eGFR threshold level.

Also provided is AP for use in a method of treating a patient having AKI, preferably sepsis associated AKI (SA-AKI) and/or AoCKI, wherein the method comprises: (a) obtaining pre-AKI eGFR values from the patient, and (b) administering an AP to the patient if the patient is determined to have a lower or decreased pre-AKI eGFR compared to a predetermined eGFR threshold level; or suspending, not initiating, or denying the administration of an AP to the patient if the patient is determined to have a higher or increased pre-AKI eGFR in the sample compared to a predetermined eGFR threshold level.

The disclosure also provides a method of determining the efficacy or pharmacodynamics of an AP in a patient diagnosed with AKI, preferably sepsis associated AKI (SA-AKI) and/or AoCKI, comprising: (a) conducting a first measurement of the patients' eGFR in a first sample taken from the patient; (b) administering the AP, e.g., RecAP; and (c) conducting a second measurement of eGFR in a second sample taken from the patient, wherein an increase or preservation of the eGFR in the second measurement compared to the patient's eGFR in the first measurement, indicates that the patient is responding to treatment with the AP, e.g., RecAP.

The disclosure also provides a method of determining the efficacy or pharmacodynamics of an AP in a patient diagnosed with AKI, preferably sepsis associated AKI (SA-AKI) and/or AoCKI, comprising: (a) conducting a first measurement of the patients' eGFR in a first sample taken from the patient; and (b) conducting a second measurement of eGFR in a second sample taken from the patient after the patient has been provided with the AP, e.g. RecAP, wherein an increase or preservation of the eGFR in the second measurement compared to the patient's eGFR in the first measurement, indicates that the patient is responding to treatment with the AP, e.g., RecAP.

The disclosure also provides a method of measuring the efficacy or pharmacodynamics of an AP in a patient diagnosed with AKI, preferably sepsis associated AKI (SA-AKI) and/or AoCKI, comprising: (a) conducting a first measurement of the patients' eGFR in a first sample taken from the patient; (b) administering the AP, e.g., RecAP; and (c) conducting a second measurement of eGFR in a second sample taken from the patient, wherein a decrease of the eGFR in the second measurement compared to the patient's eGFR in the first measurement, indicates that the patient is not responding to treatment with the AP, e.g., RecAP.

The disclosure also provides a method of measuring the efficacy or pharmacodynamics of an AP in a patient diagnosed with AKI, preferably sepsis associated AKI (SA-AKI) and/or AoCKI, comprising: (a) conducting a first measurement of the patients' eGFR in a first sample taken from the patient; and (b) conducting a second measurement of eGFR in a second sample taken from the patient after the patient has been provided with the AP, e.g. RecAP, wherein a decrease of the eGFR in the second measurement compared to the patient's eGFR in the first measurement, indicates that the patient is not responding to treatment with the AP, e.g., RecAP.

In some aspects, the second measurement is conducted 1, 2, 3, 4, 5, 6, 7, 8, 9, 10, 11, 12, 13, 14, 15, 16, 17, 18, 19, 20, 21, 22, 23, 24, 25, 26, 27, 28, or 90 days, or at 1, 2, 3, 4, 5, 6, 7, 8, 9, 10, 11, 12, 13, 14, 15 or 16 weeks, or at intervening times, after administering the AP, e.g., RecAP.

In certain aspects, a “loading” dose of an AP is administered to achieve a desired level of kidney function in the patient. If the AP loading dose does not affect the patient's kidney function significantly a decision could be made to discontinue treatment—e.g., to switch to an alternative therapy.

If the loading dose results in increased kidney function in the patient a decision could be made to reduce the AP dose size or frequency to a “maintenance” dose. It is important to note that the methods provided herein are guidelines for a healthcare provider to administer treatment, and the ultimate treatment decision will be based on the healthcare provider's sound judgment.

The formulation, dosage regimen, and route of administration of an AP, e.g., RecAP, can be adjusted to provide an effective amount for an optimum therapeutic response according to the method disclosed herein. With regard to the administration of an AP, the AP may be administered through any suitable means, compositions and routes known in the art. With regard to dosage regiments, a single bolus can be administered, several divided doses can be administered over time or the dose can be proportionally reduced or increased as indicated by the exigencies of the therapeutic situation.

### III. Alkaline Phosphatases (AP)

Alkaline phosphatase (AP; EC 3.1.3.1 according to IUBMB Enzyme Nomenclature), is an enzyme that catalyzes the reaction of a phosphatase monoester and H₂O to an alcohol and phosphate. Other name(s) for AP are alkaline phosphomonoesterase; phosphomonoesterase; glycerophosphatase; alkaline phosphohydrolase; alkaline phenyl phosphatase; orthophosphoric-monoester phosphohydrolase (alkaline optimum). The systemic name of AP is phosphate-monoester phosphohydrolase (alkaline optimum).

AP is a wide specificity enzyme, it also catalyzes transphosphorylations. In humans and other mammals at least four distinct but related AP are known. They are intestinal, placental, placental-like, and liver/bone/kidney (or tissue non-specific) AP. The first three are located together on chromosome 2 while the tissue non-specific form is located on chromosome 1.

The term “AP of the present disclosure” refers to an isolated alkaline phosphatase, including splice variants, isoforms, and polymorphic forms thereof. Also included are recombinant AP and chimeric AP. In a specific aspects, the AP is RecAP. The amino acid sequence of RecAP is shown in FIG. 1.

In some aspects, an AP disclosed herein has at least about 70%, at least about 75%, at least about 80%, at least about 85%, at least about 90%, at least about 95%, at least about 96%, at least about 97%, at least about 98%, or at least about 99% sequence identity to the amino acid sequence of SEQ ID NO:1.

In some aspects, the AP is a functional fragment (i.e., a fragment of the AP, e.g., AP conserving at least about 10%, at least about 20%, at least about 30%, at least about 40%, at least about 50%, at least about 60%, at least 70%, at least about 80%, or at least about 90% of the AP activity of the corresponding full length AP). In some aspects, the AP is a variant or a derivative of an AP disclosed herein.

An AP for use according to the present disclosure can be a commercial AP enzyme, or any composition comprising the AP enzyme and any means capable of producing a functional AP enzyme in the context of the current invention, such as DNA or RNA nucleic acids encoding an AP protein.

The nucleic acid encoding AP may be embedded in suitable vectors such as plasmids, phagemids, phages, (retro) viruses, transposons, gene therapy vectors and other vectors capable of inducing or conferring production of AP. Also native or recombinant micro-organisms, such as bacteria, fungi, protozoa and yeast may be applied as a source of AP in the context of the current disclosure.

AP containing compositions for use according to the present disclosure may comprise a eukaryotic AP, e.g., a mammalian AP, which may be of the types tissue non-specific AP, such as liver-bone or kidney type, or tissue specific such as placental AP, intestinal AP and placental-like AP. The latter, also known as germ cell AP, is localized to testis, thymus and certain germ cell tumors, and is closely related to both the placental and intestinal forms of AP.

In some aspects, the mammalian AP is a human or a bovine AP. Non-limiting examples of a human AP sequence can be found in the NCBI (Genpept) collection and include: NP_001622 (intestinal AP), NP_001623 (placental AP), NP_112603 (placental-like AP) or NP_000469 (tissue non-specific AP). In some aspects, the AP comprises a polymorphism. In some aspects, the AP is placental AP, placental-like AP, intestinal AP, liver/bone/kidney AP, or a combination thereof. In some aspects, the AP is recombinant AP.

From a conformational point of view, an AP roughly consists of two domains: a crown domain and an active-site domain. The active-site domain can be divided in separate parts like the catalytic residue and the three metal ion sites (Zn1, Zn2 and Mg3). From a primary structure point of view, the crown domain is flanked by the amino acids that form the active site domain. The amino acid sequence of APs and the relative positions of the catalytic and crown domain are known by the skilled person.

In some aspects of the present disclosure, the AP is an isolated or recombinant AP comprising a crown domain and a catalytic domain, wherein said crown domain and said catalytic domain are obtained from different APs and wherein at least one of said different phosphatases is a human phosphatase. In some aspects, the AP is, for example, ECAP (Escherichia coli AP) or one of the seven known BIAPs (Bovine Intestinal AP).

In some aspects, AP is an isolated or recombinant AP comprising a crown domain and a catalytic domain, wherein said crown domain and said catalytic domain are obtained from different APs and wherein the different APs are human APs. This is especially useful if the modified phosphatase is subsequently used in human therapy. AP for use in the disclosed methods can be modified, e.g., genetically modified, APs of human origin which are not or only weakly immunogenic.

A modified AP disclosed herein can be used, for example, in “in vitro” or “ex vivo” diagnostics or treatments. Such modified phosphatase may comprise, for example, a human and an E. coli AP or may be composed of a bovine and an E. coli AP.

In some aspects of the present disclosure, the AP is an isolated or recombinant AP comprising a crown domain and a catalytic domain, wherein said crown domain and said catalytic domain are obtained from different APs and wherein said crown domain is the crown domain of placental AP (ALPP) and wherein said catalytic domain is the catalytic domain of intestinal AP (ALPI). In some aspects, at least one of the different APs is a human phosphatase. In other aspects, both different APs are human phosphatases.

Domain swapped mutants suitable for the methods disclosed herein which are based on the human APs are listed in Table 1.

TABLE 1
Domains swapped alkaline phosphatase enzymes. ALPI
is intestinal AP, ALPP is placental AP, GCAP is placental-
like AP and TNAP is tissue non-specific AP.

| Catalytic domain | Crown domain | Referred to as |
|---|---|---|
| ALPI | GCAP | catALPI/crownGCAP |
| | TNAP | catALPI/crownTNAP |
| ALPP | GCAP | catALPP/crownGCAP |
| | TNAP | catALPP/crownTNAP |
| GCAP | ALPI | catGCAP/crownALPI |
| | ALPP | catGCAP/crownALPP |
| | TNAP | catGCAP/crownTNAP |
| TNAP | ALPI | catTNAP/crownALPI |
| | ALPP | catTNAP/crownALPP |
| | GCAP | catTNAP/crownGCAP |

In some aspects, the AP is a combination between the catalytic domain of ECAP or any of the human forms (ALPI, ALPP, GCAP or TNAP) with the crown domain of BIAP. Moreover, combinations of the crown domain of BIAP with the catalytic domain of ECAP or any of the human forms can also be produced.

In some aspects, the modified AP is an AP which under natural conditions are linked to the membrane of a cell via a glycosylphosphatidylinositol (GPI) anchor but which is modified such that it is no longer attached to the membrane of a cell. All isoenzymes are functionally active in the cell membrane and GPI-anchor deficient forms are not naturally present at detectable levels. Although serum AP activity has been demonstrated it is generally accepted that the enzyme is still present in shed membrane fractions or membrane vesicles. AP activity in milk is also present in fractions containing membrane vesicles. The GPI anchor is stored as a precursor molecule in the cell where it is attached to the attachment site through a transamidase. The backbone of the GPI-anchor is identical in mammals, but cell-type dependent modifications are known.

In some aspects, for treatment of human subjects, the AP is human. This is primarily due to the fact that AP forms obtained from other species may be immunogenic in human subjects and treatment could elicit immunological reactions and pathological side effects. In some subjects even lethal side effects, i.e., anaphylactic shock may occur and the risks of immunological side effects are therefore preferably minimized by use of human AP forms.

As isolation of AP from humans is not practical, human recombinant forms of AP proteins can be routinely produced in different recombinant expression platforms. However, expression and purification of GPI modified and membrane-anchored proteins is notoriously difficult; GPI proteins are difficult to separate from membranes and difficult to isolate and purify. Thus, in some aspects, the recombinant APs comprises a modification in the GPI signal sequence, wherein said modification results in a secreted AP, i.e., the AP is not attached to the cell membrane.

There is no general sequence responsible for the attachment of a GPI anchor, but there some specific consensus characteristics:
A hydrophobic stretch of amino acids at the C-terminus (at least 11 amino acids, but preferably more than 11 amino acids);
A spacer of hydrophilic amino acids (5-12 amino acids) upstream of the hydrophobic region;
GPI is attached to a small amino acid: glycine, aspartic acid, asparagine, alanine, serine or cysteine; and,
the 2 subsequent amino acids downstream of the GPI attachment site must be small amino acids and in the majority of cases they are selected from glycine, aspartic acid, asparagine, alanine, serine or cysteine.

In some aspects, the recombinant AP comprises a modification in the GPI signal sequence, wherein said modification results in a secreted AP that is biological active, i.e., it shows activity towards a biologically relevant substrate.

In some aspects, the secreted AP is a human AP. In some aspects, the secreted human AP human liver-kidney-bone phosphatase, human intestinal AP, or human placental-like alkaline phosphatase.

Based on the consensus characteristics above, a skilled person can introduce modifications, e.g., by inserting one or multiple amino acids, that would disrupt part of the consensus a result in an AP not capable to attaching a GPI anchor. Thus, in some aspects, the recombinant AP comprises a modification in the GPI signal sequence which results in a secreted AP, wherein the modification comprises a mutation or a deletion of at least one amino acid in the sequence encompassing the consensus GPI signal sequence.

In some aspects, the AP is an AP disclosed in U.S. Pat. No. 8,557,545. In some aspects, the AP is a chimeric AP or chimeric AP-like protein such as those described in in U.S. Patent Appl. Publ. No. US2017/0009216 and US2014/0193388. In some specific aspects of the present disclosure, the AP is a recombinant alkaline phosphatase comprising the catalytic domain of ALPI (intestinal alkaline phosphatase) and the crown domain of ALPP (placental alkaline phosphatase), e.g., RecAP (SEQ ID NO: 1). RecAP is also known as catALPI/crownALPP, Xinplap, and SALPI-ALPP-CD. In some aspects, the AP is an improved RecAP, e.g., LVL-RecAP (corresponding to SEQ ID NO:1 in U.S. Patent Appl. Publ. No. US2017/0009216).

In some aspects, an AP of the present disclosure comprises (i) a sequence having at least about 70%, at least about 75%, at least about 80%, at least about 85%, at least about 90%, at least about 95%, at least about 96%, at least about 97%, at least about 98%, or at least 99% sequence identity with the crown domain of a human ALPP, and (ii) a sequence having at least about 70%, at least about 75%, at least about 80%, at least about 85%, at least about 90%, at least about 95%, at least about 96%, at least about 97%, at least about 98%, or at least 99% sequence identity with the catalytic domain of a human ALPI.

In some aspects, said sequence having said sequence identity to the crown domain of ALPP is situated in a protein according to the invention at approximately the same position as the crown domain of ALPP in the native ALPP protein

The percentage of identity of an amino acid or nucleic acid sequence, or the term “% sequence identity”, is defined herein as the percentage of residues in a candidate amino acid or nucleic acid sequence that is identical with the residues in a reference sequence after aligning the two sequences and introducing gaps, if necessary, to achieve the maximum percent identity. In a preferred embodiment, the calculation of said at least percentage of sequence identity is carried out without introducing gaps. Methods and computer programs for the alignment are well known in the art, for example “Align 2” or the BLAST service of the National Center for Biotechnology Information (NCBI).

### EXAMPLES

### Example 1—REVIVAL

### Human Recombinant Alkaline Phosphatase for Sepsis-Associated Acute Kidney Injury

### Methods

### Overall Design

The REVIVAL trial was a Phase 3, multicenter, randomized, double-blind, placebo-controlled, 2-arm parallel group-sequential design pivotal trial in which patients with SA-AKI were randomly assigned in a 1:1 ratio to placebo or to 1.6 mg/kg RecAP.

Randomization was stratified by:
‘Moderate’ chronic kidney disease (CKD), defined as a pre-AKI reference eGFR ≥25 and <45 mL/min/1.73 m²;
Baseline Modified Sequential Organ Failure Assessment (mSOFA) score, i.e., excluding the Glasgow Coma Scale (GCS) part (≤9, >9)
Clinical site

Based on this, 3 distinct SA-AKI trial populations were defined:
1. The main trial population: Patients with a pre-AKI reference eGFR ≥45 mL/min/1.73 m² and no proven or suspected COVID-19 at time of randomization;
2. A ‘moderate’ CKD population: Patients with a pre-AKI reference eGFR 22 25 and <45 mL/min/1.73 m² and no proven or suspected COVID-19 at time of randomization
3. A COVID-19 population: Patients with proven or suspected COVID-19 at time of randomization with or without ‘moderate’ CKD and, for patients in this population, COVID-19 should have been the main cause of SA-AKI.

For each of the three populations, an independent randomization list has been produced

Patients were enrolled at approximately 100 sites predominantly across Europe, North America, Japan, and Australia. The final number of patients to be enrolled was set to depend on the recommendations of the data monitoring committee (DMC) based on the safety data reviews and interim analyses for futility/success. The individual trial flow for the patients is shown in FIG. 1.

### Inclusion Criteria

To be eligible for this trial, a patient must meet all of the following five inclusion criteria:
1. 18 years or older.
2. In the ICU or intermediate care unit for clinical reasons.
3. Have sepsis requiring vasopressor (norepinephrine, epinephrine, dopamine, phenylephrine, vasopressin, or angiotensin II) therapy, i.e.:
   a. suspected or proven bacterial or viral infection, and
   b. on vasopressor therapy (>0.1 μg/kg/min norepinephrine or equivalent) for sepsis-induced hypotension for at least one hour despite adequate fluid resuscitation according to clinical judgement. Following the initial one hour on at least 0.1 μg/kg/min norepinephrine or equivalent, any dose of vasopressor counts as vasopressor therapy.

The combination of a) and b) automatically ensures that patients fulfill the Sepsis-3 criteria as 0.1 μg/kg/min norepinephrine corresponds to a score of +4 on the Cardiovascular sub-score of the SOFA score.
4. Have AKI according to at least one of the below Kidney Disease: Improving Global Outcomes (KDIGO) criteria, a to d:
   a. An absolute increase in serum or plasma creatinine (CR) by ≥0.3 mg/dL (>26.5 μmol/L) within 48 hours.
   b. A relative increase in CR to ≥1.5 times the pre-AKI reference CR value, which is known or presumed to have occurred within prior 7 days.
   c. A decrease in urinary output to <0.5 mL/kg/hour for a minimum of 6 hours following adequate fluid resuscitation.
   d. If the patient does not have a known history of CKD and there is no pre-AKI reference CR value available from the past 12 months: a CR value greater or equal to the levels presented in Table 2, with the increase in CR presumed to have occurred within prior 7 days.
5. Provision of signed and dated informed consent form (ICF) in accordance with local regulations.

TABLE 2
Gender and Race Corrected Cut-off Values for Serum or Plasma
CR Based on 1.5 Times Estimated Normal Values for Age Group.

| | Black males | Other males | Black females | Other females |
|---|---|---|---|---|
| Age | mg/dL | mg/dL | mg/dL | mg/dL |
| (years) | (μmol/L) | (μmol/L) | (μmol/L) | (μmol/L) |
| 20-24 | 2.3 (200) | 2.0 (173) | 1.8 (159) | 1.5 (132) |
| 25-29 | 2.3 (200) | 1.8 (159) | 1.7 (146) | 1.5 (132) |
| 30-39 | 2.1 (186) | 1.8 (159) | 1.7 (146) | 1.4 (120) |
| 40-54 | 2.0 (173) | 1.7 (146) | 1.5 (132) | 1.4 (120) |
| 55-65 | 2.0 (173) | 1.7 (146) | 1.5 (132) | 1.2 (107) |
| >65 | 1.8 (159) | 1.5 (132) | 1.4 (120) | (107) |

### Exclusion Criteria

A patient who meets any of the following criteria is excluded from participation in this trial:
1. Documented CKD as specified below:
   a) At selected sites where enrolment of ‘moderate’ CKD patients is allowed:
‘Severe’ CKD defined as a pre-AKI reference eGFR <25 mL/min/1.73 m².
   For patients with known CKD, the most recent eGFR prior to index hospitalization needs to be documented as ≥25 mL/min/1.73 m².
   For patients with known CKD but no known eGFR prior to hospitalization, presentation eGFR between 25-60 mL/min/1.73 m² can also be used to rule out ‘severe’ CKD.
   b) At all other sites:
‘Moderate’ and ‘severe’ CKD defined as a pre-AKI reference eGFR <45 mL/min/1.73 m².
   For patients with known CKD, the most recent eGFR prior to index hospitalization needs to be documented as >45 mL/min/1.73 m².
   For patients with known CKD but no known eGFR prior to hospitalization, presentation eGFR between 45-60 mL/min/1.73 m² can also be used to rule out ‘moderate’ and ‘severe’ CKD.
2. Advanced chronic liver disease, defined as a Child-Pugh score of 10 to 15 (Class C).
3. Acute pancreatitis with no established source of infection.
4. Urosepsis related to suspected or proven urinary tract obstruction.
5. Main cause of AKI not sepsis.
6. Proven or suspected SARS-CoV-2 infection. This exclusion criterion does not apply to patients in the COVID-19 population.
7. Severe burns requiring ICU treatment.
8. Severely immunosuppressed, e.g. due to:
   hematopoietic cell transplantation within past 6 months prior to Screening or acute or chronic graft-versus-host disease;
   solid organ transplantation;
   leukopenia not related to sepsis, i.e., preceding sepsis;
   Human Immunodeficiency Virus (HIV)/Acquired Immune Deficiency Syndrome (AIDS);
   receiving chemotherapy within 30 days prior to screening.
9. At high risk of being lost to follow-up (LTFU), e.g., due to known current or recent (within the last 6 months) IV drug abuse or known to be homeless.
10. Limitations to use of mechanical ventilation (MV), RRT or vasopressors and inotropes (NOTE: limitation of cardiopulmonary resuscitation (CPR) only is not an exclusion criterion).
11. Previous administration of recAP.
12. Use of a non-marketed drug within the last month or concurrent or planned participation in a clinical trial for a non-marketed drug or device. (NOTE: Co-enrollment or concurrent participation in observational, non-interventional trials using no protocolized treatments or procedures are always allowed. Co-enrollment or concurrent participation in trials using protocolized treatments or procedures, e.g. blood draws, requires pre-approval by the TSC).
13. Current or planned extracorporeal membrane oxygenation (ECMO).
14. On RRT >24 hours before start of trial drug.
15. No longer on vasopressor therapy at time of randomization.
16. On continuous vasopressor therapy for >72 hours before start of trial drug.
17. Estimated glomerular filtration rate (eGFR)>60 mL/min/1.73 m² based on the most recent available CR sample at time of screening (NOTE: will often be the sample used to diagnose AKI). eGFR should be calculated using the Chronic Kidney Disease Epidemiology Collaboration (CKD-EPI) formula.
18. Not feasible to start trial drug within:
   a) 48 hours from AKI diagnosis, when AKI diagnosis precedes start of vasopressor therapy, or
   b) 24 hours from AKI diagnosis, when AKI is diagnosed after start of vasopressor therapy.
19. Pregnant or nursing women.

### Trial Drug, Dosage and Route of Administration

Trial drug (recAP or placebo) is provided in glass vials as a concentrate for infusion (aqueous buffer at a pH of 7.0). Prior to administration, the trial drug will be diluted with sterile sodium chloride 0.9% for injection (isotonic saline), USP/EP or equivalent, to a final volume of 50 mL and administered as an IV infusion using a dosing syringe or infusion bag. The intended recAP dose is 1.6 mg (1,000 U) per kg of patient body weight. Patients with a body weight >120 kg will be administered a fixed dose of 192 mg.

Trial drug (recAP or placebo) was administered as a 1-hour continuous IV infusion on Day 1, Day 2 and Day 3 by qualified staff in the ICU or intermediate care unit. The first infusion starts as soon as feasible after randomization of the patient on Day 1. On Day 2 and Day 3, trial drug administration should start 24+/−2 hours after the previous trial drug administration. In case the patient has been discharged from the ICU or intermediate care unit to a ward within the hospital before completing the last trial drug administration on Day 3, trial drug should be administered at the ward by qualified personnel following instructions provided by the trial team.

The preferred route for trial drug administration is through a central catheter; if not feasible, a peripheral line is acceptable. Trial drug will be administered separately from any other concomitant drugs using a dedicated lumen of the catheter.

### Randomization Blinding

Eligible patients are assigned a unique patient identification number via Interactive Response Technology (IRT) randomly allocating each patient to active or placebo according to the randomization schedule generated by a validated computer program. Details of the procedure are described in the IRT Manual provided to all sites.

Patients are randomly assigned 1:1 to receive either recAP or placebo.

Randomization for the main trial population and the ‘moderate to severe’ CKD population will be stratified by:
‘Moderate to severe’ CKD defined as a pre-AKI reference eGFR >25 and <45 mL/min/1.73 m²
   Yes, No
Baseline mSOFA score, i.e., excluding the GCS part.

$$mSOFA score \leq 9$$

$$mSOFA score > 9$$

Clinical site

Randomization for the COVID-19 population will be stratified by:
Baseline mSOFA score, i.e., excluding the GCS part.

$$mSOFA score \leq 9$$

$$mSOFA score > 9$$

Clinical site

### Relevant Endpoints

The following endpoints, were measured:

90-Day all-Cause Mortality;
MAKE 90: dead or on RRT until and including D28 or on RRT at D90; ≥25% decline in estimated glomerular filtration rate (eGFR) on Day 90 relative to the known or assumed pre-AKI reference level; and/or rehospitalization by D90;
Adverse Events.

### Analysis

### Analysis Populations:

Overall Efficacy Population: All patients who are randomly assigned to a trial drug and for whom administration of trial drug was started. One overall combined population consisting of the main trial population, the ‘moderate to severe’ CKD population and the COVID-19 population. All patients were analyzed according to the treatment they received regardless of what their planned treatment was. One treated subject without any efficacy data has been excluded from the efficacy analysis.

Overall Safety Population: All patients who are randomly assigned to a trial drug and for whom administration of trial drug was started. One overall combined population consisting of the main trial population, the ‘moderate to severe’ CKD population and the COVID-19 population. All patients were analyzed according to the treatment they received regardless of what their planned treatment was.

### Efficacy Analyses

### Mortality Day 90;

Absolute and relative frequencies of patients who died prior to day 90 have been analyzed. All data available for patients who were lost to follow-up prior to day 90 were included in the event rate analysis.

Kaplan-Meier estimates of the time to mortality up to day 90 (inclusive day 90) have been generated. The difference in proportions in the survival rate has been tested using the Wald test for differences in proportions. Patients who dropped out prior to day 90 have been censored.

Make90; Make 90 was defined as dead or on RRT until and including D28 or on RRT at D90 or ≥25% decline in estimated glomerular filtration rate (eGFR) on Day 90 relative to the known or assumed pre-AKI reference level or rehospitalization by D90.

Absolute and relative frequencies of patients with MAKE90 events have been analyzed. All data available for patients who were lost to follow-up prior to day 90 were included in the event rate analysis. Analysis has been done on the overall population as well as differentiated for 15 ml increments of the pre-AKI reference eGFR values of the subjects.

Kaplan-Meier estimates of the time first MAKE90 event (inclusive day 90) have been generated. The difference in proportions in the survival rate has been tested using the Wald test for differences in proportions. For the assessment, whether subjects where on RRT at day 90, or whether a 25% drop in eGFR was reported at day 90, the information from the visit documentation has been used also in cases whether the visit was not exactly on day 90. Patients who dropped out prior to day 90 have been censored.

A logistic regression of MAKE90 events was performed in order to analyze the interaction between pre-AKI reference eGFR and treatment received. The model contained MAKE90 as outcome variable of interest as well as treatment, pre-AKI reference eGFR and pre-AKI reference eGFR by interaction as factors.

Safety Analyses: Adverse Events; Incidence of AEs and serious AE were summarized by trial drug group. Event rates have been compared by using a chi-squared test.

### Results

### Participants

Among 676 enrolled patients, there were 21 (3.1%) screen failures, the majority being due to trial protocol deviation (i.e., inclusion/exclusion criterion was not met) reported in 16 (2.4%) patients (see Table 3). 655 where randomized and 650 patients have been treated with either ilofotase alfa 1.6 mg/kg (recAP 1.6 mg/kg) or placebo.

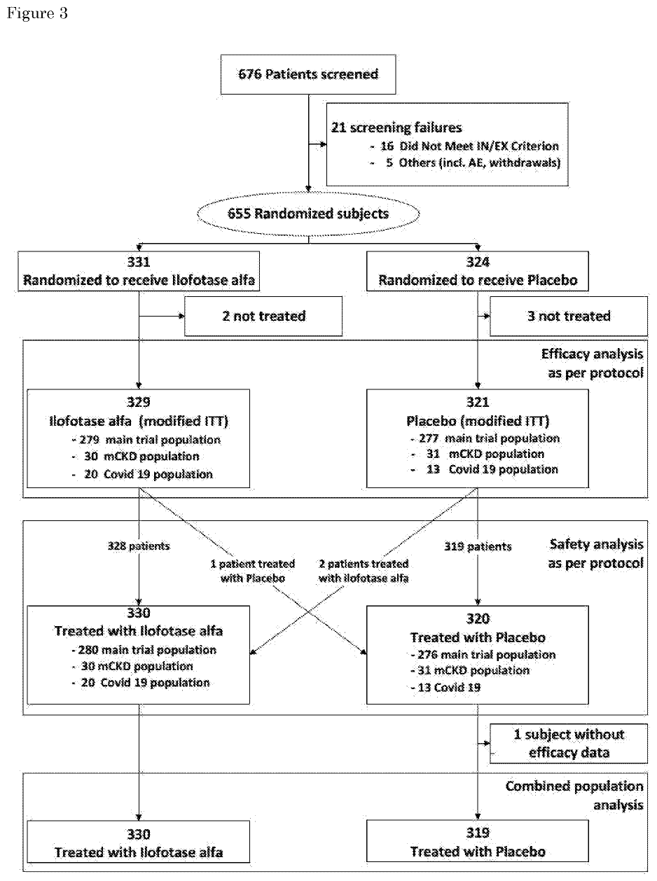

The overall efficacy population included 649 patients, 330 subjects in the ilofotase alfa arm and 319 in the placebo arm (FIG. 3). One subject has been excluded from efficacy analysis due to missing efficacy data.

The overall safety population included 650 patients, 330 subjects in the ilofotase alfa arm and 320 in the placebo arm (FIG. 3).

### Efficacy Outcomes

90 Day all-Cause Mortality

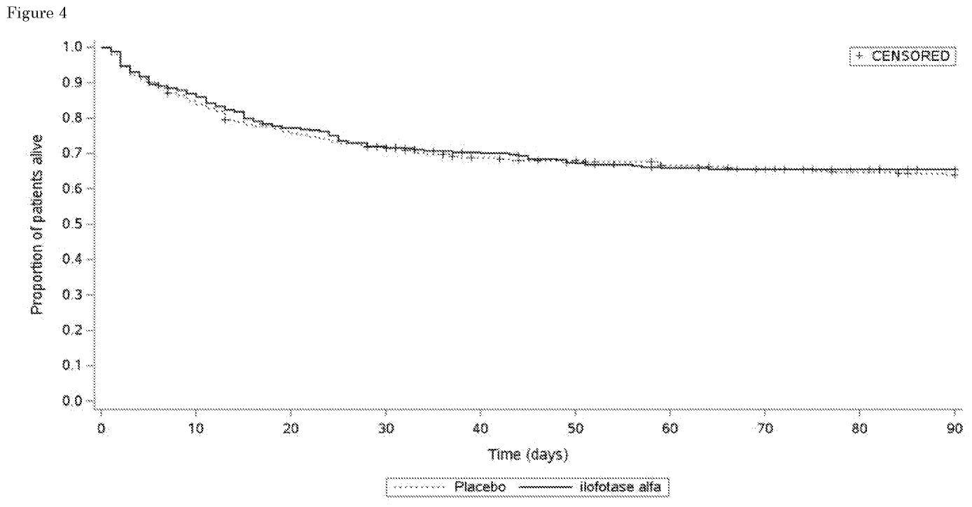

For mortality up to and including day 90, the proportion of patients that died in the placebo group was 34.8% compared to 33.9% in the ilofotase alfa group (Table 4). The Kaplan-Meier survival rate estimate at day 90 was 65.54% for the ilofotase alfa group and 63.97% for the placebo group. The estimated mortality rates are 34.46% (1-0.6554) in the ilofotase alfa group and 36.03% (1-0.6397) in the placebo group. No statistical difference could be detected using the Wald test for differences in proportions of patients alive at day 90 (p=0.3404) (FIG. 4).

### MAKE90

For MAKE90, the proportion of patients with a MAKE90 event was 56.7% in the ilofotase alfa group compared to 64.6% in the placebo group (Table 5).

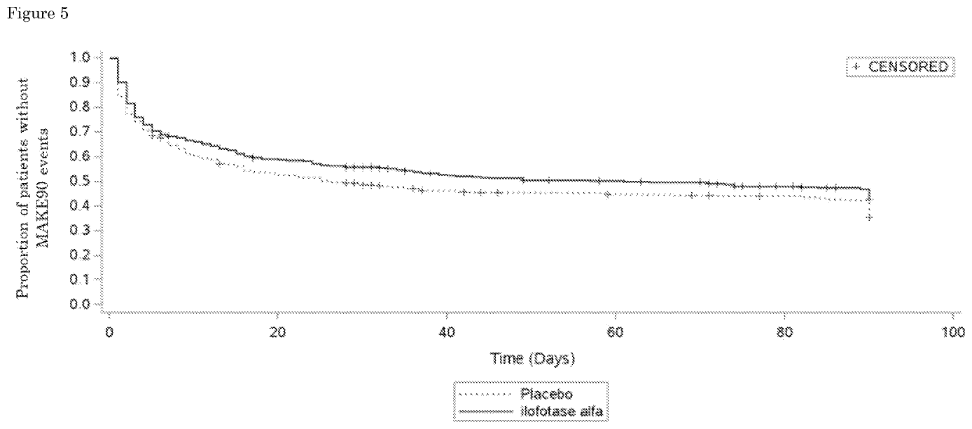

The Kaplan-Meier survival rate, i.e., the proportion of patients without an MAKE90 event, was estimated 42.77% for the ilofotase alfa group and 35.33% for the placebo group. The estimated rates of subjects with a MAKE90 event are 57.23% (1−0.4277) in the ilofotase alfa group and 64.7% (1−0.3533) in the placebo group. Using the Wald test for differences in proportions of patients without MAKE90 events, a significant difference in favor to ilofotase alfa could be detected (p=0.031) (FIG. 5.).

To further define the potential benefit for patients with lower pre-AKI reference eGFR receiving ilofotase alfa, MAKE90 was also investigated using subgroups defined by increments of pre-AKI reference eGFR equal to 15 mL/min/1.73 m². The following subgroups have been analyzed: <=30, 30 to <=45, 45 to <=60, 60 to <=75, 75 to <=90 and >90. The respective observed number and percentage of patients with MAKE90 events by treatment received are presented in Table 6. From this table, it can be seen that a lower proportion of patients receiving ilofotase alfa experienced MAKE90 events compared with patients receiving placebo in all subgroups where the pre-AKI reference eGFR was <=90 mL/min/1.73 m². However, the difference was less in the >75 to <=90 mL/min/m² subgroup.

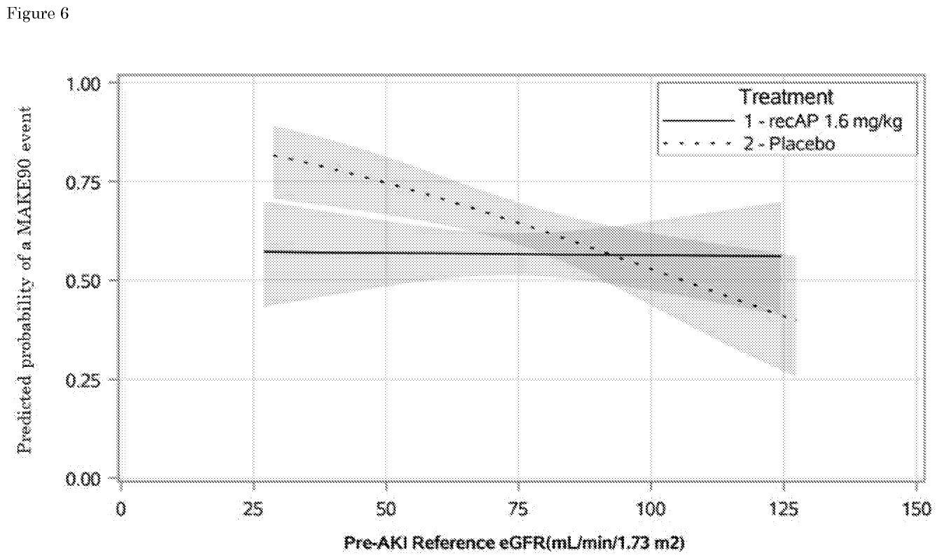

This effect has been further substantiated by performing a logistic regression on the MAKE90. The model contained MAKE90 as outcome variable of interest as well as treatment, pre-AKI reference eGFR and pre-AKI reference eGFR by interaction as factors. The treatment effect on MAKE90 was observed significant in favor for ilofotase alfa (p=0.068). The pre-Aki reference eGFR (p=0.0174) and the respective interaction between treatment and pre-AKI reference eGFR (0.0235) was also significant, suggesting a higher benefit for subjects with a lower pre-AKI-reference eGFR when comparing ilofotase alfa vs. placebo (Table 7). This effect can be visualized by plotting the predicted probability of an Make 90 event relative to the pre-AKI reference eGFR (FIG. 6).

Make90 in Subgroups with Respect to Pre-AKI Reference eGFR
Pre-AKI Reference eGFR <60

For MAKE90, the proportion of patients with an Make 90 event was 59.3% in the ilofotase alfa group compared to 81.3% in the placebo group (Table 8).

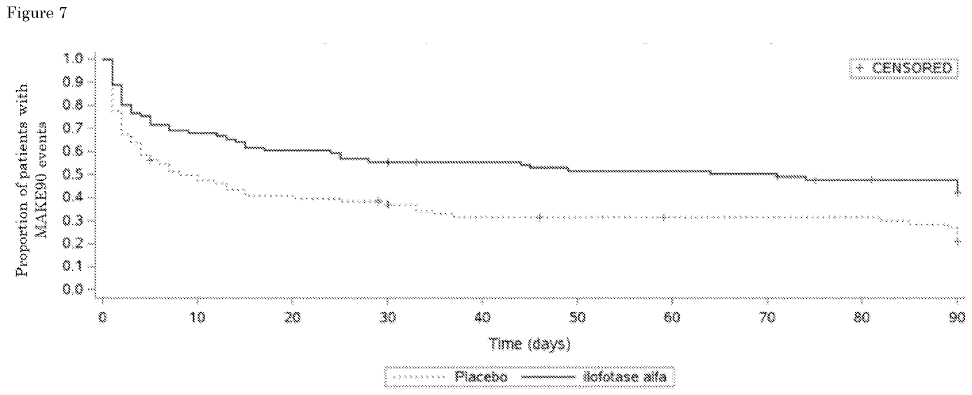

The Kaplan-Meier survival rate, i.e proportion of patients without a MAKE90 event, was estimated 42.15% for the ilofotase alfa group and 21.03% for the placebo group. The estimated rates of subjects with a MAKE90 event are 57.85% (1-0.4215) in the ilofotase alfa group and 78.97% (1-0.2103) in the placebo group. Using the Wald test for differences in proportions of patients without MAKE90 events, a significant difference in favor to ilofotase alfa could be detected (p=0.002) (FIG. 7).

Pre-AKI Reference eGFR <75

For MAKE90, the proportion of patients with an Make 90 event was 56.6% in the ilofotase alfa group compared to 71.3% in the placebo group (Table 9).

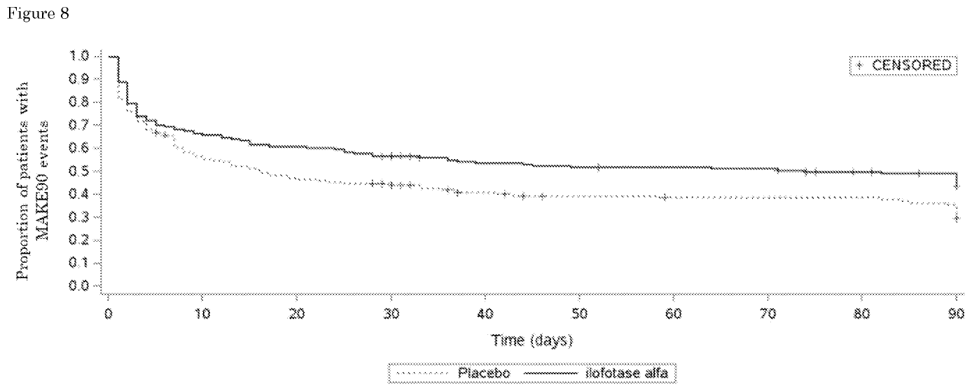

The Kaplan-Meier survival rate, i.e proportion of patients without a MAKE90 event, was estimated 43.61% for the ilofotase alfa group and 29.71% for the placebo group. The estimated rates of subjects with a MAKE90 event are 56.39% (1-0.0.4361) in the ilofotase alfa group and 70.29% (1-0.2971) in the placebo group. Using the Wald test for differences in proportions of patients without MAKE90 events, a significant difference in favor to ilofotase alfa could be detected (p=0.005) (FIG. 8).

### Safety Outcomes

### Adverse Events

A brief overview of the AEs among the safety set for the combined population is presented in Table 10. The proportions of patients with AEs (67.9% for ilofotase alfa and 75.0% for Placebo) are statistically significant (p=0.0446).

### CONCLUSION

A significantly lower proportion of patients receiving ilofotase alfa are estimated to experience MAKE90 events after the start of trial drug compared with patients receiving placebo (57.2% for ilofotase alfa and 64.7% for placebo; p-value for the difference ilofotase alfa minus placebo=0.031). This effect was most pronounced in patients with pre-existent renal repairment.

Overall, ilofotase was well tolerated and no safety concerns were identified.

TABLE 3
Overview of Trial Populations and Screen Failures (all enrolled).

| | Total |
|---|---|
| Patients | n (%) |
| All Enrolled Analysis Set^{a} | 676 |
| Screening Failures^{b, c} | 21 (3.1%) |
| Primary Reason | |
| Adverse Event | 2 (0.3%) |
| Did Not Meet Inclusion/Exclusion Criterion | 16 (2.4%) |
| Withdrawal by Legally Authorized Representative | 1 (0.1%) |
| Withdrawal by Physician | 1 (0.1%) |
| Withdrawal by Patient | 0 |
| Other | 1 (0.1%) |
| ITT Sets^{b, d} | 655 (96.9%) |
| Main Trial Population | 560 (82.8%) |
| ‘Moderate to severe’ CKD Population | 62 (9.2%) |
| COVID-19 Population | 33 (4.9%) |

Abbreviations: CKD = chronic kidney disease; COVID-19 = Corona Virus disease 2019; ITT = intent-to-treat.
^{a}All patients that had been assigned a patient number regardless of whether they were randomized or received trial drug.
^{b}Percentages are calculated based on ‘All Enrolled Analysis Set’.
^{c}Patients who had consent to participate but who were not subsequently randomly allocated to trial drug.
^{d}All patients who were randomly assigned to trial drug.

TABLE 4
Overall day 90 Mortality by treatment.

| | Treatment Group | | | | | |
|---|---|---|---|---|---|---|
| | Placebo (N = 319) | | ilofotase alfa (N = 330) | | Total (N = 649) | |
| | Subgroup | | | | | |
| | Number | Number (%) | Number | Number (%) | Number | Number (%) |
| | of subjects | of subjects | of subjects | of subjects | of subjects | of subjects |
| | in subgroup | with event | in subgroup | with event | in subgroup | with event |
| Overall | 319 | 111 (34.8%) | 330 | 112 (33.9%) | 649 | 223 (34.4%) |

TABLE 5
MAKE90 and MAKE90 Components by treatment received.

| | Treatment Group | | |
|---|---|---|---|
| | Placebo | ilofotase | Total |
| | (N = 319) | alfa (N = 330) | (N = 649) |
| | Subgroup | | |
| | Number (%) | Number (%) | Number (%) |
| | of subjects | of subjects | of subjects |
| | with event | with event | with event |
| MAKE90 | 206 (64.6%) | 187 (56.7%) | 393 (60.6%) |
| >25% drop in eGFR at Day 90 compared | 28 (8.8%) | 19 (5.8%) | 47 (7.2%) |
| with pre-AKI reference eGFR | | | |
| On RRT at Day 90 OR on RRT until and | 116 (36.4%) | 93 (28.2%) | 209 (32.2%) |
| including Day 28 | | | |
| Death up to and including Day 90 | 111 (34.8%) | 112 (33.9%) | 223 (34.4%) |
| Rehospitalisation | 30 (9.4%) | 28 (8.5%) | 58 (8.9%) |

TABLE 6
MAKE90 by treatment and increments of pre-AKI reference eGFR equal to 15.

| | Number (%) of patients with event | | | |
|---|---|---|---|---|
| | Placebo | | ilofotase alfa | |
| | (N = 319) | | (N = 330) | |
| MAKE90 * | | 206 (64.6%) | | 187 (56.7%) |
| pre-AKI reference eGFR | N in | | N in | |
| (mL/min/1.73 m²) ** | subgroup | | subgroup | |
| <=30 | 5 | 5 (100%) | 3 | 2 (66.7%) |
| >30 to <=45 | 18 | 13 (72.2%) | 24 | 13 (54.2%) |
| >45 to <=60 | 57 | 47 (82.5%) | 54 | 33 (61.1%) |
| >60 to <=75 | 92 | 58 (63.0%) | 92 | 50 (54.3%) |
| >75 to <=90 | 74 | 40 (54.1%) | 82 | 43 (52.4%) |
| >90 | 73 | 43 (58.9%) | 75 | 46 (61.3%) |

Only patients with a non-missing pre-AKI reference eGFR are included.
* Percentages are based on the number of patients within each treatment group
** Percentages are based on the number of patients in the subgroup

TABLE 7
Logistic regression analysis of MAKE90 - assessing the interaction
between pre-AKI reference eGFR and treatment received.

| | Ilofotase |
|---|---|
| Placebo | Alfa |

| | | |
|---|---|---|
| Number of patients in analysis set | 319 | 330 |

| | | | | |
|---|---|---|---|---|
| Number of patients with confirmed | 206 | (64.6%) | 187 | (56.7%) |
| MAKE90 | | | | |

Patients without confirmed MAKE90

| | | | | |
|---|---|---|---|---|
| Number of patients confirmed as | 64 | (20.1%) | 75 | (22.7%) |
| not having MAKE90 | | | | |
| Number of patients with missing | 33 | (10.3%) | 42 | (12.7%) |
| data at Day 90 | | | | |
| Number of patients with unknown | 16 | (5.0%) | 26 | (7.9%) |
| data at Day 90 | | | | |

Results for logistic regression model
P-values for co-variates used in the model

| Treatment | 0.0068 |
|---|---|
| Pre-AKI reference eGFR [mL/min/1.73 m²] | 0.0174 |
| Interaction between treatment and pre-AKI | 0.0235 |
| reference eGFR [mL/min/1.73 m²] | |

TABLE 8
MAKE90 and Make 90 components in patients
with Pre-AKI reference eGFR <60.

| | Treatment Group | | |
|---|---|---|---|
| | Placebo | Ilofotase | Total |
| | (N = 80) | alfa (N = 81) | (N = 161) |
| | Subgroup | | |
| | Number (%) | Number (%) | Number (%) |
| | of subjects | of subjects | of subjects |
| | with event | with event | with event |
| MAKE90 | 65 (81.3%) | 48 (59.3%) | 113 (70.2%) |
| Death up to and including Day 90 | 31 (38.8%) | 25 (30.9%) | 56 (34.8%) |
| >25% drop in eGFR at Day 90 compared | 9 (11.3%) | 7 (8.6%) | 16 (9.9%) |
| with pre-AKI reference eGFR | | | |
| On RRT at Day 90 OR on RRT until and | 39 (48.8%) | 22 (27.2%) | 61 (37.9%) |
| including Day 28 | | | |
| Rehospitalisation | 10 (12.5%) | 7 (8.6%) | 17 (10.6%) |

TABLE 9
MAKE90 and MAKE90 components in patients
with Pre-AKI reference eGFR <75.

| | Treatment Group | | |
|---|---|---|---|
| | Placebo | Ilofotase | Total |
| | (N = 171) | alfa (N = 173) | (N = 344) |
| | Subgroup | | |
| | Number (%) | Number (%) | Number (%) |
| | of subjects | of subjects | of subjects |
| | with event | with event | with event |
| MAKE90 | 122 (71.3%) | 98 (56.6%) | 220 (64.0%) |
| Death up to and including Day 90 | 65 (38.0%) | 53 (30.6%) | 118 (34.3%) |
| >25% drop in eGFR at Day 90 compared | 14 (8.2%) | 14 (8.1%) | 28 (8.1%) |
| with pre-AKI reference eGFR | | | |
| On RRT at Day 90 OR on RRT until and | 75 (43.9%) | 48 (27.7%) | 123 (35.8%) |
| including Day 28 | | | |
| Rehospitalisation | 17 (9.9%) | 14 (8.1%) | 31 (9.0%) |

TABLE 10
Overall summary of adverse events (combined main, covid
and mCKD population sets, treated analysis set).

| | Actual Treatment Group | | |
|---|---|---|---|
| | Placebo | Ilofotase | |
| | (N = 320) | Alfa (N = 330) | |
| | n (%) | n (%) | P-value* |
| Number of patients with adverse events | 240 (75.0%) | 224 (67.9%) | 0.0446 |
| Number of patients with serious adverse events | 141 (44.1%) | 143 (43.3%) | 0.8514 |
| Number of patients with fatal adverse events | 98 (30.6%) | 99 (30.0%) | |
| Number of patients with serious non-fatal adverse events | 62 (19.4%) | 64 (19.4%) | |
| Number of patients with drug-related adverse events | 32 (10.0%) | 30 (9.1%) | |
| Number of patients with serious drug-related adverse events | 16 (5.0%) | 16 (4.8%) | |
| Number of patients with adverse events leading to withdrawal | 6 (1.9%) | 9 (2.7%) | |
| of trial drug | | | |
| Number of patients with serious adverse events leading to | 5 (1.6%) | 8 (2.4%) | |
| withdrawal of trial drug | | | |

Number of patients with adverse events by severity**

| | | |
|---|---|---|
| Mild | 31 (9.7%) | 29 (8.8%) |
| Moderate | 59 (18.4%) | 48 (14.5%) |
| Severe | 150 (46.9%) | 147 (44.5%) |

*P-value derived from a Chi-squared test.
**The severity of an AE is assessed by an investigator as mild, moderate or severe. Cases with unknown severity is assumed to be severe.
**Patients are counted by their worst adverse event category, which is severity followed by moderate and mild.
Percentages are calculated using total number of patients per treatment group as the denominator.

1. A method for treating acute kidney injury (AKI) in a subject in need thereof, comprising administering an effective amount of alkaline phosphatase (AP) to said subject, wherein the subject has acute-on-chronic kidney injury, and wherein the chronic kidney injury (CKI) is mild, moderate, or severe.

2. The method of claim 1, wherein pre-AKI estimated glomerular filtration rate (eGFR) is ≤75 ml/min/1.73 m².

3. The method of claim 1, wherein pre-AKI eGFR is >15 ml/min/1.73 m².

4. The method of claim 1, wherein the administration of AP results in a decreased risk of developing major adverse kidney events by day 90 (MAKE90), with respect to the risk of MAKE90 in the absence of treatment.

5. The method of claim 4, wherein the MAKE90 comprises one or more of the following events: (i) death before or on day 90, (ii) renal replacement therapy before or at day 90, (iii) ≥25% drop in eGFR at day 90 relative to pre-AKI eGFR, and (iv) rehospitalization before or at day 90.

6. The method of claim 1, wherein the AP is administered in at least one 500 U/kg to 2,000 U/kg dose.

7. The method of claim 1, wherein the AP is a human AP.

8. The method of claim 1, wherein the AP is a recombinant AP.

9. The method of claim 1, wherein AP is administered once daily or in three daily doses.

10. The method of claim 1, wherein AP is administered intravenously.

11. The method of claim 1, wherein the AP dose is about 1.6 mg/kg of RecAP and/or about 1000 U/kg of RecAP.

12. The method of claim 1, wherein the administration of at least one dose of AP results in the preservation at day 90 of glomerular filtration rate (GFR) or estimated GFR (eGFR) in the subject, relative to pre-AKI GFR or eGFR.

13. The method of claim 1, wherein AKI is due to or accompanied by sepsis.

14. The method of claim 1, wherein treatment is initiated within 96 hours after sepsis is detected and/or within 48 hours after acute kidney injury (AKI) is detected in said subject.

15. The method of claim 1, wherein a decreased eGFR due to chronic kidney injury has been determined at least 15 days, at least 30 days, at least 60 days, or at least 90 days prior to the administration of AP.

16. The method of claim 1, wherein pre-AKI eGFR is ≤60 ml/min/1.73 m².

17. The method of claim 1, wherein pre-AKI eGFR is ≥25 ml/min/1.73 m².

18. The method of claim 1, wherein the recombinant AP is chimeric.

19. The method of claim 1, wherein the chimeric AP has at least 80%, at least 85%, at least 90%, at least 95%, at least 96%, at least 97%, at least 98%, at least 99%, or 100% sequence identity to the amino acid sequence of SEQ ID NO: 1.


SEQUENCE LISTING (1 sequence)

SEQ ID NO: 1 - protein, 484 aa, synthetic construct
VIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEILRDPTLDPSLMEMTEAALRLLSRNPRGFYLFVEGGRIDHGHHEGVAYQAVTEAVMFDDAIERAGQLTSEEDTLTLVTADHSHVFSFGGYPLRGSSIFGLAPGKARDRKAYTVLLYGNGPGYVLKDGARPDVTESESGSPEYRQQSAVPLDEETHGGEDVAVFARGPQAHLVHGVQEQSFVAHVMAFAACLEPYTACDLALPACTTD